(19) United States
(12) Patent Application Publication
(10) Pub. No.: US 2024/0424215 A1
(43) Pub. Date: Dec. 26, 2024

(54) ADD-ON DEVICE FOR AN INJECTION DEVICE

(71) Applicant: Sanofi, Paris (FR)

(72) Inventors: Beate Franke, Frankfurt am Main (DE); Tim Glässer, Rüsselsheim (DE); Michael Helmer, Frankfurt am Main (DE); Stephan Mücke, Rüsselsheim (DE); Peter Nober, Rüsselsheim (DE)

(21) Appl. No.: 18/700,924

(22) PCT Filed: Nov. 1, 2022
(86) PCT No.: PCT/EP2022/080410

§ 371 Date: Apr. 12, 2024

(30) Foreign Application Priority Data
Nov. 3, 2021 (EP) 21315211.9

(51) Int. Cl. A61M 5/20; A61M 5/315

(52) U.S. Cl. CPC A61M 5/2033; A61M 5/315; A61M 2005/2006; A61M 2205/3327; A61M 2205/3576; A61M 2205/502; A61M 2205/581; A61M 2205/583; A61M 2205/6045

(57) ABSTRACT

The present disclosure relates to an add-on device for attachment to an injection device. The injection device includes a device housing and a drive mechanism. The device housing accommodates a medicament container filled with a medicament. The drive mechanism operably engages with the medicament container to dispense a dose of the medicament. The injection device includes at least one code. The add-on device includes: a fastener, a code recognition unit, a logic circuit, a processor and a communication interface. The fastener attaches the add-on device to the device housing. The a code recognition unit detects and/or captures the at least one code of the injection device. The logic circuit, the processor and the communication interface execute at least one electronically implemented function of the add-on device for assisting a user in operating the injection device and/or for monitoring operation of the injection device.

### CROSS REFERENCE TO RELATED APPLICATIONS

The present application is the national stage entry of International Patent Application No. PCT/EP2022/080410, filed on Nov. 1, 2022, and claims priority to Application No. EP 21315211.9, filed on Nov. 3, 2021, the disclosures of which are incorporated herein by reference.

### TECHNICAL FIELD

The present disclosure relates to an add-on device for attachment to an injection device. The add-on device is configured to assist a user in operating the injection device and/or for monitoring operation of the injection device by the user. In further aspect the disclosure relates to an injection device, a kit of at least a first and a second injection device and to a drug delivery system as well as to a method of configuring an add-on device.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, are conditioned to meet a number of user-specific requirements. For instance, with patients suffering chronic diseases, such as diabetes, the patient may be physically infirm and may also have impaired vision. Suitable drug delivery devices especially intended for home medication therefore are configured to be robust in construction and to be easy to use. Furthermore, manipulation and general handling of the device and its components are configured to be intelligible and easy understandable. Such injection devices should provide setting and subsequent dispensing of a dose of a medicament of variable size. Moreover, a dose setting as well as a dose dispensing procedure can be optimized to facilitate operation and clarity of usage.

A patient suffering from a particular disease may require a particular amount of a medicament to either be injected via a pen-type injection syringe or infused via a pump.

Some drug delivery or injection devices provide selecting of a dose of a medicament of variable size and injecting a dose previously set. Other injection devices provide setting and dispensing of a fixed dose. For example, the amount of medicament that should be injected in accordance to a given prescription schedule is always the same and does not change or cannot be changed over time.

Some injection devices are implemented as reusable injection devices offering a user to replace a medicament container, such as a cartridge. Other injection devices are implemented as a disposable injection device. With disposable injection devices it is intended to discard the entirety of the injection device when the content, e.g., the medicament, has been used up.

In order to control and to supervise medication conducted by users or patients themselves it is desirable to provide an automated detecting and logging of a repeated and regular use of the drug delivery device. Especially for fixed dose injection devices typically offering a simple and rather effective approach for delivery of many drug therapies, recording of doses injected by a user would offer a significant advantage over a manual dose logging in terms of security and convenience.

Attachment of an add-on device to an injection device can be advantageous, both in terms of providing a reliable monitoring of user operations conducted with the injection device as well as for providing a high degree of user acceptance. Attachment of an add-on device to the injection device can be easy to implement and rather intuitive.

Some scenarios of using the injection device include use of a kit of injection the devices comprising at least a first injection device and a second injection device. The injection devices may comprise a similar if not identical shape but are equipped with medicament containers comprising different volumes, comprising different medicaments or comprising different concentrations of a pharmaceutically active component. For example, for each injection device there may be required an individual add-on device.

### SUMMARY

This disclosure relates to an add-on device for attachment to an injection device. The add-on device can be universally usable with a series of injection devices. User acceptance for using the add-on device can be improved. The add-on device can further provide an easy mechanical assembly to the injection device and vice versa.

In one aspect the disclosure relates to an add-on device for attachment to an injection device. The injection device comprises a device housing. The device housing may comprise a first housing component and a second housing component. The first and the second housing component may complement to form the housing of the injection device. Typically, the first housing component is configured to accommodate a medicament container filled with a medicament, typically a liquid injectable medicament. The second housing component is configured to support or to house, e.g., to accommodate a drive mechanism. The drive mechanism is configured to operably engage with the medicament container for dispensing or expelling of a dose of the medicament from the medicament container. Typically, the drive mechanism comprises a piston rod operable to exert a pressure onto a piston of the medicament container.

The add-on device comprises a fastener for attaching the add-on device to the device housing. The add-on device further comprises a code recognition unit operable to detect and/or to capture the at least one code of the injection device. The add-on device further comprises a logic circuit, a processor and a communication interface operable to execute at least one electronically implemented function of the add-on device for assisting a user in operating the injection device and/or for monitoring operation of the injection device. For example, at least one of an execution and a calibration of the at least one electronically implemented function is modifiable by a signal obtainable from the code recognition unit in response to detect and/or to capture the at least one code of the injection device.

The code of the injection device may be automatically detected by the code recognition unit before, during or after attaching or fastening the add-on device to the device housing. With some examples an automatic code detection or code capturing is obtained in the course of fastening or attaching the add-on device to the device housing.

The fastening of the add-on device is configured for engagement with a complementary-shaped counter fastener of the device housing of the injection device. The fastener and the counter fastener may form a clip joint, a formfitting joint, a biomed joint or a friction joined. The fastener may be clamped on a dedicated portion of the housing of the injection device. The fastener may engage with a proximal end of the device housing, with a distal end of the device housing or with an intermediate section of the device housing located between a distal end and the proximal end of the device housing.

The code of the injection device is provided at a dedicated portion of the housing of the injection device such that a detection or capturing of the code is possible when the add-on device is correctly fastened to the injection device.

With the code recognition unit, the add-on device can be individually adapted to a specific injection device provided with a specific code. The code provides an identifier to identify a specific type of an injection device. By reading or capturing of the code the add-on device can be automatically adapted to the detected type of the injection device.

For instance, an injection device of a first type and being provided with a code of a first type may have to be operated in a different way than an injection device of a second type and being provided with a second code.

By detecting or capturing the code by the code recognition unit the add-on device can be adjusted, calibrated or modified so as to adapt to the specific demands of the injection device. On the basis of the code and the code recognition, injection device-specific information can be selected and provided to a user through the communication interface. By way of the code and the code recognition the add-on device, e.g., a monitoring function of the add-on device can be adjusted or calibrated accordingly.

Typically, the code recognition unit is connected or coupled to the processor of the add-on device. This way, the processor can be provided with respective code information as provided by the code on or in the housing of the injection device. Upon recognition of a code by the code recognition unit the processor may automatically deploy or select a suitable software for use with that particular type of injection device or drive mechanism that corresponds to the detected code.

The code recognition unit may be implemented electrically, electronically and/or electro-mechanically. With a mechanical implementation of the code recognition unit comprising a movable component, the degree or magnitude of the movement of the movable component of the code recognition unit is electronically detectable. For example, the degree or magnitude of the movement of the movable component is provided as an electronic signal to the processor.

With other types of the code recognition unit, the code recognition unit provides an electronic signal being unequivocally indicative of a counter coding or code provided on one of the first and second housing components.

With an injection device of a first type a movement of a device component, e.g., a movement of a component of the drive mechanism may correspond to setting or dispensing of a dose of a first size. With another type of injection device, a similar or identical detection of a respective movement of that particular device component may represent a second dose setting or dose injection that distinguishes from the first dose setting or dose injection. By way of the code and the code recognition the add-on device can be calibrated and/or adjusted accordingly.

The code on the injection device and the code recognition unit of the add-on device provides the benefit that a single add-on device can be universally used with a variety of drug delivery devices or injection devices of different type. For example, a user that may use different types of injection devices can make use of one and the same add-on device, that may be interchangeably used with different injection devices of a set or kit of injection devices.

In this way, a dosing history or medication history of the respective patient making use of a variety of injection devices of a set or kit of injection devices can be acquired and monitored by one and the same add-on device. With the code and the code recognition unit a precise and reliable adjustment of the add-on device can be provided every time when an injection device of a first type is interchanged by another injection device, e.g., of a second type.

The electronically implemented function of the add-on device may comprise or include any data processing or signal processing function of the add-on device. For instance, an electronically implemented function of the add-on device may comprise processing of signals obtainable a sensor of the add-on device, which signals being indicative of at least one of setting of a dose and dispensing of a dose of the medicament by the injection device when the add-on devices assembled to the injection device. For example, the sensor signals may quantitatively specify a degree or magnitude of a movement or position of a movable component of the injection device, which position or movement relative to e.g., a housing of the injection device or fastener of the add-on device is indicative of a size of a dose currently set or dispensed by the injection device.

With further examples the electronically implemented function of the add-on device includes generation of at least one of an audible or visible output in response to detect or to capture the at least one code of the injection device.

According to a further example the at least one code provided on the injection device is indicative of at least one of a type of the medicament, a concentration of a pharmaceutically active substance of the medicament, a size of the medicament container, a type of the drive mechanism, a manufacturing date of the medicament, an expiry date of the medicament or combinations thereof.

Depending on the information acquired from the code recognition the add-on device may be configured to conduct numerous functions. For instance and depending on the type of a medicament or depending on the concentration of the medicament the add-on device may calibrate a dose setting or dose monitoring function.

Depending on the detectable code and hence depending on at least one of the type of the medicament, the concentration of the pharmaceutically active substance or the particular drive mechanism the add-on device is configured to provide respective medicament specific and/or drive mechanisms specific information to the user by or through the communication interface.

For instance, an injection device of a first type is provided with a first type of patient information. An injection device of a second type is provided with a second type of patient formation. This way and depending on the detected code the appropriate information, e.g., stored in a electronic storage of the add-on device can be selected and provided to the user of the add-on device via the communication interface.

Different types of drive mechanisms may require different steps of operation. By detecting a code of the housing being indicative of the specific type of a drive mechanism the processor may select a respective information from the memory and is further operable to provide the specific information of the detected drive mechanism to the user. In a similar way, different drive mechanisms may require different types of signal processing obtainable from a sensor of the add-on device.

According to a further example the add-on device comprises at least one sensor connected to the processor and operable to detect and/or to quantitatively measure at least one of setting of a dose and dispensing of a dose of the medicament by the injection device. By way of the sensor a user-induced operation of the injection device can be detected and/or quantitatively measured.

The add-on device may be further equipped with a clock enabling to log a time of use or operation of the add-on device. For example, a date and time of a dose setting operation as well as the date and time of a dose injection performed or conducted by the injection device can be monitored and logged. Typically, the add-on device is provided with an electronic memory connected to the processor. By way of the memory a sequence of repeated operations of the injection device can be stored locally in the add-on device. The electronic memory further provides storage of different configurations or functions of the add-on device. When the add-on device is intended for use with differently configured drive mechanisms, e.g., intended for use with medicament containers of different type a suitable calibration of the sensor for recording and/or measuring dose setting and/or dose injecting events can be provided in the electronic memory.

According to a further example the code recognition unit of the add-on device is connected to the processor. The processor is operable to calibrate and/or to modify at least one electronically implemented function of the add-on device on the basis of an identification signal obtainable from the code recognition unit. The identification signal is indicative of a specific type of at least one of a plurality of available injection devices.

In this way, the add-on device can be electronically configured in accordance to the code as provided on one or in the device housing. Upon recognition of the code by the code recognition unit the processor may automatically deploy or select a suitable software for use with that particular type of drive mechanism that corresponds to the detected code. By an automatic identification of one of a plurality of available drive mechanisms through the code the add-on device is implemented to automatically select the correct type and/or the correct software routine for signal processing.

The code recognition unit may be implemented electrically, electronically and/or electro-mechanically. With a mechanical implementation the code recognition unit comprising a movable component, the degree or magnitude of the movement of the movable component of the code recognition unit is electronically detectable. The degree or magnitude of the movement of the movable component is provided as an electronic signal to the processor. With other types of the code recognition unit, there is provided an electronic signal being unequivocally indicative of the code provided on or in the housing of the injection device.

For instance the processor is operable to calculate a size of a dose on the basis of signals obtained from the sensor and on the basis of the detected or captured code. Calculation of a size of a dose may be regarded as an example of an electronically implemented function of the add-on device. Typically, the sensor is operable to detect or to quantitatively measure a degree of movement of a movable component of the drive mechanism, e.g., during setting of a dose or during injecting of the dose. With a drive mechanism of a first type, a first degree of movement of a movable component is correlated to a first dose size. With another drive mechanism the same degree of movement of the movable component is indicative of a second dose size that differs from the first dose size. This might be due to a different volume, e.g., a different diameter of the medicament container.

By detecting the type of the drive mechanism through the interaction of the code and the code recognition unit the detectable movement of the movable component of the drive mechanism can be precisely matched with the correct dose size. Hence, the detected code provides a calibration or adjustment of the dose size recognition or dose size calculation provided by the add-on device.

According to a further example the processor is operable to generate at least one of an audible or visible output in response to detect or to capture the at least one code of the injection device. In a first approach the processor may simply provide a visual identification on a display of the add-on device upon recognition of a particular type of injection device. Generation of at least one audible or visible output may be regarded as a further example of an electronically implemented function of the add-on device.

Here, the user may be provided with relevant information regarding the particular type of injection device. As an example, the user may be provided with the name of a drug, with the concentration of the pharmaceutically active substance, with a size of the medicament container as well as with further information about the medicament, such as the manufacturing date or the expiry date of the medicament.

Depending on the information gathered by code recognition the add-on device may automatically trigger an alert or an approving signal to the user. In situations, in which the add-on device may e.g., detect that the expiry date of the medicament has already lapsed, the processor is operable to generate a respective alert, either audibly or visibly by a speaker or by a display of the add-on device. A user of the add-on device or injection device is then immediately provided with a respective alert not to use the medicament. Generation of an element are of an approving signal may be regarded as a further example of an electronically implemented function of the add-on device.

Further functions of the processor may include a confirmation to the user, e.g., that a particular amount of a medicament has been set by the injection device or has been dispensed or injected by the injection device.

According to a further example the communication interface comprises a transceiver to communicate with an external electronic device. The external electronic device is typically provided at least with one of a display and a speaker.

Communication between the communication interface of the add-on device and the external electronic device may be implemented by a wireless communication link. The wireless communication link as well as a wire-based communication link can be generally used to provide visual and/or audible feedback or information to the user via the external electronic device. Hence, the add-on device itself may be void of a display and/or void of a speaker. For example, at least one of a display and a speaker as provided by the external electronic device may be used instead. This remote use is enabled by the communication link between the add-on device and the external electronic device.

Typically, the external electronic device is a portable electronic device. The external electronic device may be implemented as a smartphone, as a smart watch or as a tablet computer.

According to a further example, the add-on device comprises a mounting adapter. The mounting adapter is configured for fastening the add-on device to the device housing. The mounting adapter comprises a first connecting interface and a second connecting interface. The first connecting interface is connectable to a first housing component. The second connecting interface is connectable to a second housing component. In this way the mounting adapter is configured for arrangement in an interface between the first housing component and the second housing component.

This provides several benefits in terms of a mutual mechanical fixing of the add-on device and the injection device. First of all, the mounting adapter provides an elegant approach to arrange and to attach the add-on device to the device housing. Arrangement of the mounting adapter of the add-on device in the interface between the first and the second housing component may provide a rather attractive design of the drug delivery system comprising the add-on device and the injection device.

Arrangement of the mounting adapter between first and second housing components of the injection device may also provide a rather stable and long-lasting mechanical fixing of the mounting adapter to the device housing.

Assembling the add-on device and hence the mounting adapter to the first housing component and to the second housing component may enforce a presence and indispensable use of the mounting adapter and hence of the add-on device in order to enable a mutual connection and/or fixing of the first housing component to the second housing component; and vice versa. In other words, the mounting adapter may be required to mutually assemble and fix the first housing component and the second housing component.

With some examples the first housing component is only indirectly connectable to the second housing component via the mounting adapter; and vice versa. In this way use of the injection device is only possible with the mounting adapter assembled between the first and the second housing components. Without the mounting adapter and hence without the add-on device a mutual assembly of the first housing component and the second housing component may be effectively prevented. In this way, it can be guaranteed or enforced, that the injection device can only be used in connection with the add-on device.

According to a further example the first connecting interface is connectable to a proximal connecting end of the first housing component. The second connecting interface is connectable to a distal connecting end of the second housing component. With some examples the distal connecting end of the second housing component may be directly connectable to the proximal connecting end of the first housing component. With other examples the distal connecting end is incompatible with the proximal connecting end. For example, a mutual assembly of the first and second housing components requires use of the mounting adapter in the interface between the first and second housing components.

According to a further example the mounting adapter comprises a body with a distal end provided with the first connecting interface and with a proximal end opposite to the distal end.

The proximal end of the body of the mounting adapter is provided with the second connecting interface. The mounting adapter, in particular its body, may comprise an adapter sleeve comprising a diameter or cross-section towards the distal end that matches a cross-section or diameter of the proximal connecting end of the first housing component. Likewise, the proximal end of the body of the mounting adapter may comprise a diameter or cross-section that matches the respective diameter or cross-section of the distal connecting end of the second housing component. In this way a somewhat nested or interleaved mutual assembly configuration of the mounting adapter with at least one of the proximal connecting end and the distal connecting end can be provided.

According to another example one of the first connecting interface and the second connecting interface comprises an interface insert configured for insertion into a housing receptacle of one of the first housing component and the second housing component. The other one of the first connecting interface and the second connecting interface comprises an interface receptacle configured to receive a housing insert of the other one of the first housing component and the second housing component.

According to the described implementation, an at least partially nested or interleaved configuration between the first connecting interface and the first housing component and/or between the second connecting interface and the second housing component can be provided. Such an interleaved or nested configuration is beneficial in terms of providing a rather stable, long-lasting and robust mutual interconnection between the mounting adapter and the first and second housing components.

With some examples it is the first housing component that comprises a housing insert configured for insertion into an interface receptacle provided at the first connecting interface. With other examples the proximal end of the first housing component comprises a housing receptacle shaped and configured to receive a complementary shaped interface insert provided at the first connecting interface. The same or a similar configuration is conceivable for the connection between the second connecting interface at the second housing component.

With some examples the second connecting interface comprises an interface insert configured and shaped for insertion into a complementary shaped housing receptacle of the second housing component. With other examples it is the second connecting interface that comprises an interface receptacle shaped and sized to receive a housing insert provided on the distal end of the second housing component.

With some examples the first connecting interface comprises an interface receptacle and the second connecting interface comprises an interface insert. With other examples the first connecting interface and the second connecting interface each comprise an interface insert configured and shaped for insertion into respective housing inserts of the first housing component and the second housing component, respectively. For example, a mutual assembly of the first housing component and the second housing component without the mounting adapter is effectively impeded because the first housing component and the second housing component each comprise a housing receptacle, providing a female connector. For example, the add-on device is provided with complementary shaped interface inserts. With a further example, the add-on device comprises two interface receptacles on each of the first and second connecting interface complementary shaped to a housing insert provided on each of the first and second connecting end.

With other examples, the first connecting interface and the second connecting interface comprise one interface insert and one interface receptacle. For example, the first housing component comprises one of a complementary shaped housing insert and a housing receptacle and the second housing component comprises the other one of a complementary shaped housing insert and housing receptacle.

With some examples, the add-on device and hence the mounting adapter comprises one female connector, e.g., in form of an interface receptacle and one male connector, e.g., in form of an interface insert. The first housing component and the second housing component may be directly connectable and/or fixable by way of mutually corresponding male and female connecting structures, such as a housing insert provided on one of the first and the second housing component complementary shaped to a housing receptacle of the other one of the first and a second housing component.

The presence of mutually corresponding or complementary shaped mechanical connecting structures, such as an insert and a receptacle on the first and second connecting interfaces as well as on the first and second housing components provides a rather robust, long-lasting and stable mutual assembly and fixing of first and second housing components with the mounting adapter.

According to another example the first connecting interface comprises a first fastening element configured to engage with a complementary shaped first counter fastening element of the first housing component. The second connecting interface comprises a second fastening element configured to engage with a complementary shaped second counter fastening element of the second housing component. The first fastening element and the first counter fastening element are mutually engageable so as to fasten and to fix the first connecting interface and hence the add-on device to the first housing component. The second fastening element and the complementary shaped second counter fastening element are operable to mutually fasten and fix the mounting adapter and hence the add-on device to the second housing component.

According to a further example one of the first fastening element and the first counter fastening element comprises a protrusion. The other one of the first fastening element and the first counter fastening element comprises a recess complementary shaped to the protrusion.

With a further example one of the second fastening element and the second counter fastening element comprises a protrusion. The other one of the second fastening element and the second counter fastening element comprises a recess complementary shaped to the protrusion. Complementary shaped protrusions and recesses provided on the connecting ends of the housing components and the connecting interfaces of the mounting adapter provide a form fitting engagement of the fastening element with the respective counter fastening element. A form fitting engagement, wherein the protrusion engages a complementary-shaped recess provides a rather stable, robust and well-defined mutual fastening and fixing of the mounting adapter to the respective housing component.

According to a further example the first fastening element and the first counter fastening element and/or the second fastening element and the second counter fastening element form one of a clip joint, a threaded joint and a bayonet joint between the mounting adapter and the respective first or second housing component.

According to another example the first fastening element of the first connecting interface is incompatible with the second fastening element of the second connecting interface. Correspondingly, the first counter fastening element of the first housing component complementary shaped to the first fastening element is incompatible with the second counter fastening element complementary shaped to the second fastening element. In this way it is effectively impeded that the first and second housing components can be directly and mutually fastened because the first counter fastening element of the first housing component does not match the second counter fastening element of the second housing component.

According to a further example at least one of the first connecting interface and the second connecting interface comprises a coding. The coding is configured to cooperate with a complementary shaped counter coding of one of the first housing component and the second housing component. By way of a coding and a complementary shaped counter coding there may be provided different types of injection devices. For example, a first type of injection device may be equipped with a with a first type of a medicament container. A second type of an injection device may be equipped with a second type of a medicament container. Medicament containers of different types may distinguish by their medicament, by the amount of the medicament contained inside the medicament container as well as by a concentration of a pharmaceutical active component of the medicament. For each type of a medicament or medicament container there may be provided an individually configured drive mechanism.

By way of a coding provided on one of the first housing component and a complementary shaped counter coding of a second housing component an unintended cross use of a housing component non-matching with another housing component can be prevented. With mutually corresponding pairs of a coding and a counter coding provided on the first and the second housing components it can be guaranteed that only a first housing component equipped with a first type of a medicament container can be used in connection with a second housing component equipped with a corresponding first type of a drive mechanism. Non-matching pairs of a coding and a counter coding prevent a mutual fastening of the first housing component and the second housing component. Unintended cross use of first and second housing components not intended to be combined can be effectively prevented.

With at least one of the first connecting interface and the second connecting interface being provided with a coding, e.g., with a coding of a first type, a connection with one of the first and second housing component provided with a counter coding of a second type, e.g., a non-matching coding, can be effectively prevented. In this way, it can be guaranteed, that only one of the first and second housing components intended for use with the add-on device can be connected with a respective first or second connecting interface of the add-on device.

With the present terminology the code of the injection device is provided on an outside surface of the device housing or inside the device housing, whereas the counter coding is provided on one of the connecting ends of the first housing component and the second housing component respectively. Both, the code and the counter coding are detectable and/or capturable by the add-on device in order to distinguish the type of injection device from other types of injection devices. The code provided on or in the injection device may coincide with a counter coding.

With some examples the code may be provided in addition to a counter coding of at least one of the first and the second housing components of the injection device.

According to a further example the first connecting interface of the add-on device comprises a first coding. The first coding is configured to cooperate with a first counter coding of the first housing component. Additionally or alternatively, the second connecting interface of the add-on device comprises a second coding configured to cooperate with a second counter coding of the second housing component.

With some examples the first counter coding of the first housing component and the second counter coding of the second housing component mutually match. For example, the first counter coding may be complementary shaped to the second counter coding so that the first housing component can be directly connected with the second housing component. With other examples the first counter coding is incompatible with the second counter coding. For example, the add-on device will be required to mutually connect the first housing component and the second housing component.

According to a further example the coding comprises a mechanical coding feature. The counter coding comprises a complementary shaped mechanical counter coding feature. With mutually corresponding mechanical coding features and mechanical counter coding features a mutual fastening of one of the first connecting interface and the second connecting interface with a respective first and/or second housing component can be effectively prevented when the mechanical coding features do not match with the mechanical counter coding features.

Mechanical coding features of a first type may distinguish from mechanical coding features of a second type e.g., by the number of coding features, by a longitudinal position of coding features relative to a housing component or relative to one of the first and second connecting interfaces, by a longitudinal extent of the coding features, by a circumferential and/or radial position of the coding features relative to the housing component or connecting interface, by a respective circumferential and/or radial extent of coding features, a cross-sectional geometry or shape of the coding features in a plane transverse to the longitudinal direction.

There may be provided different types of mechanical coding features, e.g., male coding features and female coding features. Male coding features may comprise a longitudinal or radial protrusion whereas female coding features may comprise a longitudinal or radial recess complementary shaped to the respective male coding feature.

According to a further example connecting the first connecting interface to the first housing component is blocked and/or impeded when the first coding does not match the first counter coding. Additionally or alternatively, connecting of the second connecting interface to the second housing component is blocked and/or impeded when the second coding does not match with the second counter coding.

With a further example the mechanical coding feature comprises at least one of a coding protrusion and a coding recess. It is defined by at least one of a number of coding features on a sidewall of the first connecting interface, a longitudinal position, a longitudinal extent, a circumferential position, a circumferential extent and/or by a cross-sectional geometry or shape of the coding protrusion and/or of the coding recess in a plane transverse to a longitudinal direction of the first or second housing component in a plane transverse to a longitudinal direction of the mounting adapter.

Coding protrusions and coding recesses of a mechanical coding and of a mechanical counter coding that that do not match are operable to prevent a mutual fastening of the first and second housing components equipped with respective male and female mechanical coding features. Moreover, non-matching coding protrusions and coding recesses of a coding and a counter coding of the different type prevent a mutual fastening of one of the first and second connecting interfaces with a respective first or second housing component.

According to a further example the mechanical coding feature is defined by a position or orientation of the at least one coding protrusion or coding recess relative to the at least one fastening element. In the same way, the mechanical counter coding feature may be defined by a position or orientation of the at least one counter coding protrusion or counter coding recess relative to the at least one counter fastening element. Different types of first and second housing components may be provided with a common shape or configuration of counter fastening elements. The same may apply to the fastening elements of the mounting adapter. When there are provided different mounting adapters with different codings the respective mounting adapters may be provided with a common configuration or common shape of fastening features.

With a further example of the code recognition unit is operable to detect and/or to capture a counter coding provided on or in one of the first housing component and the second housing component. The code recognition unit allows and supports implementation of further types of mechanical codings and mechanical counter codings. The code recognition unit may be operable to detect and/or to capture an electric or electronic coding, a magnetic coding or a visual or optical coding and/or combinations thereof. In this way, the variety of how to implement a coding on the mounting adapter and/or to implement a counter coding on at least one of the first and second housing components can be increased.

According to a further example the second connecting interface comprises the code recognition unit, which is operable to detect and/or to capture the second counter coding provided on or in the second housing component. The add-on device is provided with a functionality to detect a particular type of a drive mechanism housed or supported by the second housing component. With a code recognition unit provided on the second connecting interface, the second connecting interface may be void of a second coding. Here it may be sufficient when the second counter coding as provided on the second housing component is detected and/or captured by the code recognition unit.

With another example it is the first connecting interface of the add-on device that comprises the code recognition unit, which is operable to detect and/or to capture the first counter coding provided on or in the first housing component. For example, the first counter coding may be void of a mechanical coding. Moreover, the first connecting interface may be void of a first coding complementary shaped to the first counter coding of the first housing component.

With either implementation of the code recognition unit the connecting interface of the add-on device equipped with the code recognition unit may be void of a respective coding. For example, the particular counter coding to be detected or to be captured by the code recognition unit may be implemented mechanically or non-mechanically. With the second connecting interface provided with the code recognition unit any type of differently coded second housing component may be connected or may be connectable with the mounting adapter.

The same may be valid for examples wherein the first connecting end of the mounting adapter is provided with the code recognition unit. Also here, each type of differently encoded first housing components is connectable and fastenable to the first connecting interface of the mounting adapter provided that the first connecting interface is equipped with the code recognition unit.

According to a further example the coding of at least one of the first connecting interface and the second connecting interface is a reconfigurable mechanical coding. Typically, the reconfigurable mechanical coding is provided on that one of the first and second connecting interface that is void of the code recognition unit. Typically, when one of the first and second connecting interfaces is provided with the code recognition unit the other one of the first and the second connecting interfaces is provided with a reconfigurable mechanical coding.

A reconfigurable mechanical coding provides a universal adaptation and modification of a mechanical coding. In this way it can be provided that only one type of an add-on device and that only one type of a mounting adapter can be fitted to several if not all types of differently coded first and second housing components. For instance, when a second housing component is provided with a second counter coding of a first type and when the code recognition unit is provided on the second connecting end a mutual connection of the mounting adapter with the second housing component provides an automated recognition or capturing of the second counter coding.

The add-on device may then comprise a first mechanical coding at the first connecting interface implemented as a reconfigurable mechanical coding. for example, the reconfigurable mechanical coding may be subject to a configuration or reconfiguration so that only a first housing component provided with a first mechanical counter coding of the first type can be attached and connected to the mounting adapter. A first mechanical counter coding of a second type will be effectively prevented from connecting to the mounting adapter and hence to the add-on device.

According to a further example the add-on device comprises a coding configurator operably connected to the code recognition unit. The coding configurator is operable to configure or to adapt the reconfigurable mechanical coding to a complementary-shaped counter coding detected and/or captured by the code recognition unit. The coding configurator may be implemented mechanically, electronically and/or electro-mechanically. With a mechanical implementation the coding configurator may comprise a follower movably disposed in or on the mounting adapter. The follower may be subject to a movement or displacement upon connecting, e.g., the second housing component to the second connecting interface. The follower may be further mechanically engaged with the reconfigurable mechanical coding. The reconfigurable mechanical coding, e.g., provided at the first connecting interface may be subject to a reconfiguration depending on the degree of movement or displacement of the follower.

With other examples and when implemented electronically or electro-mechanically the coding configurator may comprise an electromechanical actuator, which is controlled by a processor of the add-on device. Typically, the electromechanical actuator is operated and/or controlled in accordance to the type of a mechanical counter coding as detected or captured by the code recognition unit. The code recognition unit may be operable to generate electric signals to be processed by the processor, wherein the electric signals of the code recognition unit are indicative of a particular type of a counter coding provided on a respective first or second housing component.

According to a further example of the add-on device one of the first connecting interface and the second connecting interface comprises the reconfigurable mechanical coding. The other one of the first connecting interface and the second connecting interface comprises the code recognition unit. The add-on device may individually adapt to a counter coding provided on one of the first and the second housing components. A second housing component provided with a second counter coding, e.g., of a first type, may be detected by the code recognition unit as provided on the second connecting interface. Depending on the detected or captured counter coding the coding configurator adapts or reconfigures the first mechanical coding of the mounting adapter such that only a first housing component provided with a first counter coding of the first type is allowed to connect to the mounting adapter and hence to the add-on device. A first counter coding of a different type will be hindered to connect to the mounting adapter and hence to the add-on device.

Implementation of a code recognition unit on one of the first connecting interface and the second connecting interface in combination with a reconfigurable mechanical coding on the other one of the first connecting interface and the second connecting interface provides the benefit that only one add-on device and only one mounting adapter can be used with several if not all available types of different counter codings of the first and the second housing components, respectively. It will not be necessary to provide different mounting adapters or different add-on devices for different types of mechanical codings of the first and second housing components. In this way only one and the same type of an add-on device can be universally used with a variety of differently configured injection devices.

According to a further example at least one of the code recognition unit and the coding configurator comprises the follower. The follower is movably arranged in or on the housing of the add-on device. The follower is operably engageable with one of the first mechanical counter coding provided on the first housing component and the second mechanical counter coding provided on the second housing component. Furthermore, the follower is operably engaged with the configurable mechanical coding.

With some examples, the configurable mechanical coding is directly or indirectly connected to the follower. The follower is movable between at least a first follower position and a second follower position through a mechanical interaction with one of the first mechanical counter coding and the second mechanical counter coding. Typically, the add-on device is connectable with the second housing component. In the course of connecting and attaching the add-on device to the second housing component the second mechanical counter coding of the second housing component is engageable with the follower. In the course of mutual assembly of the add-on device and the second housing component the follower is subject to a movement from the first follower position, e.g., an initial position, towards and into the second follower position.

The movement of the follower is induced by a mounting or fastening of the second housing components to the add-on device, namely by the engagement of the second counter coding of the second housing component with the follower. The follower is connected or operably engaged with the configurable mechanical coding. The configurable mechanical coding is typically provided at the first connecting interface. It is mechanically engageable with the first housing component.

Depending on the degree or magnitude of the movement of the follower from the initial position towards the final assembly position, e.g., the second position, a respective movement, e.g., a respective reconfiguration of the mechanical coding can be achieved. This way, the configurable mechanical coding can be individually adapted to the second mechanical counter coding as provided on the second housing component.

In in this way, the first coding of the add-on device can be changed and reconfigured to match with a first counter coding of a first housing component intended to be used with the second housing component comprising the second mechanical coding.

In effect, by way of the code recognition unit, the configurable mechanical coding and the coding configurator the add-on device can be individually adapted to any available coded pair of first and second housing components.

Hence, a single add-on device can be used with a variety of injection devices of different type each of which comprising first and second housing components being complementary coded.

According to another aspect the present disclosure further relates to the injection device for injecting a dose of a medicament. The injection device comprises a device housing configured to accommodate a medicament container filled with the medicament. The device housing is further configured to support or to house and/or to accommodate a drive mechanism. The drive mechanism is configured to operably engage with the medicament container for dispensing, e.g., for expelling of the dose of the medicament.

The injection device further comprises at least one code on the device housing or in the device housing detectable and/or captured rebel by a code recognition unit of an add-on device as described above.

The injection device is intended for use with the above-described add-on device. Therefore, all features, effects and benefits described above in connection with the add-on device equally apply to the injection device.

According to a further example the medicament container filled with the medicament is arranged inside the first housing component. The medicament container may be implemented as a cartridge prefilled with the liquid medicament. The cartridge may comprise a tubular-shaped barrel. The tubular shaped barrel comprises a distal end provided with an outlet. The outlet may be provided with a pierceable seal, such as a septum fixed to the outlet. Towards the proximal end the barrel of the medicament container is sealed by a stopper movably disposed inside the barrel.

The drive mechanism of the injection device typically comprises a piston rod to exert a distally directed pressure onto the stopper. Under the force effect excretable by the piston rod the stopper can be moved in distal direction relative to the barrel thereby expelling a dose of the medicament via the outlet of the medicament container. With some examples the outlet of the container is penetrated by a double-tipped injection needle allowing to inject the dose of the medicament into biological tissue.

In another aspect the present disclosure further relates to a kit of at least a first injection device as described above and a second injection device as described above. The first injection device comprises a first code. The second injection device distinguishes from the first injection device by at least one of the following parameters, namely: a type of the medicament, a concentration of a pharmaceutically active substance of the medicament, a size of the medicament container, a type of the drive mechanism, a manufacturing date of the medicament, an expiry date of the medicament and combinations thereof.

The first code is indicative of any one of the above-mentioned parameters of the first injection device. The second code is indicative of any of the above-mentioned parameters of the second injection device.

The code may be implemented as mechanical code, as an electrically detectable code, as a magnetically detectable code, as a visually detectable code, as an optically detectable code or by combinations thereof. Some examples of the code may be electrically and optically detectable or capturable. Some other examples of the code may be mechanically and electrically and/or optically detectable.

With some examples the code may be characterized by the magnitude of an electrical resistance. for example, the code may comprise a first electrical contact and a second electrical contact interconnected by an electrical resistor. Different codes may distinguish from each other by the magnitude of the electrical resistor whereas the first and second electrical contact of different codes may be somewhat identical.

With some examples the code comprises a surface-mounted resistor between the first and the second electrical contacts and electrically interconnecting the first and the second electrical contacts.

By way of distinguishing different codes on the first and second injection devices the add-on device can be automatically adapted and configured to the respective injection device currently used with the add-on device.

In effect, first and second injection devices that may distinguish by one of the above-mentioned parameters can be used with one and the same add-on device.

According to another aspect the disclosure further relates to a drug delivery system. The drug delivery system comprises an injection device as described above or a kit of injection devices as described above. The drug delivery system further comprises an add-on device as described above. The add-on device may be universally usable with injection devices of different types, e.g., injection devices equipped with different codes.

According to another aspect the present disclosure further relates to a method of configuring an add-on device for attachment to an injection device. The injection device comprises a device housing and a drive mechanism. The device housing is configured to accommodate a medicament container filled with an injectable medicament. The drive mechanism is further configured to operably engage with the medicament container to dispense a dose of the medicament. The injection device comprises at least one code.

The method of configuring the add-on device comprises the steps of fastening and/or attaching the add-on device to the device housing. As a further step the method comprises detecting and/or capturing the at least one code of the injection device, typically by a code recognition unit of the add-on device. As a further step the method comprises modifying at least one of an execution and a calibration of at least one electronically implemented function of the add-on device on the basis of the detected or captured code of the injection device.

The code may be implemented mechanically, electrically, magnetically, optically or visually. The code recognition unit is a correspondingly implemented so as to detect and/or to capture the code provided on or in the injection device. Depending on the detected or captured code the add-on device can be reconfigured in accordance to the code and hence in accordance to the type of medicament, the concentration of a pharmaceutical active substance of the medicament, in accordance to a size of the medicament container, in accordance to a type of the drive mechanism and so on.

Typically, the method of configuring the add-on device is conducted by using the add-on device as described above in connection with an injection device as described above. Insofar, all features, effects and benefits as described above in connection with the add-on device, the injection device and the drug delivery system equally apply to the method of configuring the add-on device; and vice versa.

Generally, the scope of the present disclosure is defined by the content of the claims. The injection device is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g., a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034, MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g., a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multi-specific antibody fragments such as bi-specific, tri-specific, tetraspecific and multi-specific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, particular residues within the framework regions of particular antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE DRAWINGS

In the following, numerous examples of a data logging device for monitoring use of an injection device as well as a respective injection device will be described in greater detail by making reference to the drawings, in which:

FIG. 1 is indicative of a longitudinal cross-section of one example of an injection device implemented as a pen-type injector,

FIG. 2 shows an exploded view of an add-on device for use with an injection device,

FIG. 3 shows the drug delivery system of FIG. 2 in a final assembly configuration,

FIG. 4 schematically illustrates one example of first and second connecting interfaces of the mounting adapter of the add-on device connected with first and second housing components,

FIG. 5 shows a cross-section through the components of FIG. 4 in a preassembly configuration,

FIG. 6 shows one example of a mechanical counter coding of first and second housing components of a first type,

FIG. 7 shows another example of a mechanical counter coding of first and second housing components of a second type, and

FIG. 8 shows another example of a mechanical counter coding of first and second housing components of a third type,

FIG. 9 shows a perspective illustration of one example of the second connecting interface and the second housing component,

FIG. 10 is a cross-section through an example of an electrically implemented counter coding and a code recognition unit,

FIG. 11 shows another example of FIG. 10,

FIG. 12 shows another example of a counter coding pursuant to FIGS. 10 and 11,

FIG. 13 shows an example of an electronically or electrically detectable counter coding,

FIG. 14 shows another example of an electrically detectable counter coding,

FIG. 15 shows another example of an electrically detectable counter coding,

FIG. 16 shows three different examples of a counter coding provided on or in the second housing component,

FIG. 17 shows two different examples of a mechanical counter coding of the first housing component,

FIG. 18 is a block diagram of logic circuit or logic components of the add-on device,

FIG. 19 shows an example of a sensor implementation of the add-on device,

FIG. 20 shows another example of a sensor implementation of the add-on device,

FIG. 21 shows an example of the add-on device communicating with an external electronic device,

FIG. 22 shows a flowchart of a method of setting up the add-on device,

FIG. 23 shows an example of a reconfigurable mechanical coding,

FIG. 24 schematically shows a longitudinal cross-section of the first housing component connectable to the connecting interface of FIG. 23,

FIG. 25 shows a longitudinal cross-section through the connecting interface of FIG. 23,

FIG. 26 shows a longitudinal cross-section through an example of the add-on device provided with a code recognition unit and provided with a reconfigurable mechanical coding,

FIG. 27 shows an example of an electromechanically implemented coding configurator of FIG. 26,

FIG. 28 schematically illustrates implementation of an electric or electronic counter coding on the first housing component,

FIG. 29 schematically shows a longitudinal cross-section through another example of the add-on device,

FIG. 30 shows another example of the add-on device provided with a mechanically implemented coding configurator,

FIG. 30 shows an example of a first housing component,

FIG. 32 shows a side view of the housing component of FIG. 31,

FIG. 33 shows the connecting end of a second housing component,

FIG. 34 shows a side view of the housing component of FIG. 33,

FIG. 35 shows a cross-section A-A through follower sleeve of a coding configurator,

FIG. 36 shows a longitudinal cross-section through the follower sleeve,

FIG. 37 shows a cross-section B-B through the follower sleeve pursuant to FIG. 36,

FIG. 38 shows a cross-section A-A through the follower sleeve in an initial configuration,

FIG. 39 shows a cross-section B-B of the follower sleeve in the initial configuration,

FIG. 40 shows a cross-section A-A of the follower sleeve in a final assembly configuration,

FIG. 41 shows a cross-section B-B in the final assembly configuration,

FIG. 42 shows a cross-section A-A of the follower sleeve in the initial configuration,

FIG. 43 shows a cross-section B-B in the initial configuration with a different counter coding,

FIG. 44 shows a cross-section A-A in a final assembly configuration,

FIG. 45 shows a cross-section B-B in the final assembly configuration, and

FIG. 46 shows a flowchart of reconfiguring a mechanical coding.

### DETAILED DESCRIPTION

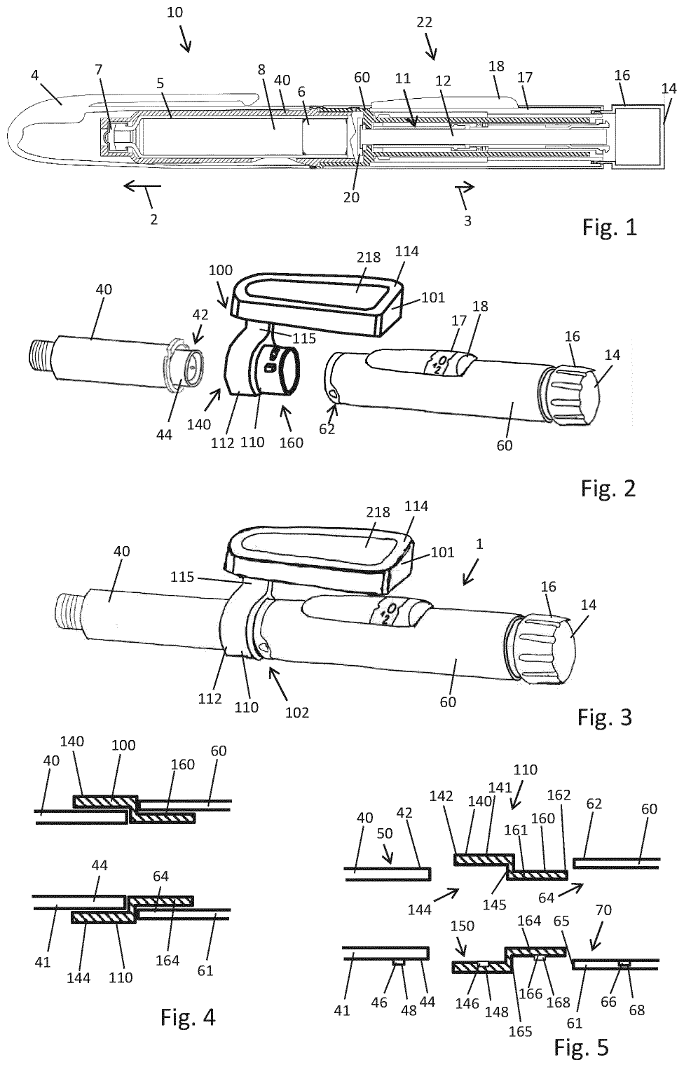

In FIG. 1 a longitudinal cross-section of one example of an injection device 10 is schematically illustrated. The injection device 10 is implemented as a pen-type injector. The injection device 10 may be implemented as a reusable device. It may be also implemented as a disposable device intended to be discarded, e.g., when the medicament container is empty.

The injection device 10 comprises a device housing 22. The device housing 22 comprises a first housing component 40, also denoted as a distal housing component and a second housing component 60 also denoted as a proximal housing component. The injection device 10 comprises a drive mechanism 11. The drive mechanism 11 comprises a piston rod 12 comprising a pressure piece 20 at a distal end. The piston rod 12 is configured to advance in a distal direction 2 and to exert pressure on a stopper 6 of a medicament container 5 located inside the first housing component 14.

The medicament container 5 comprises a tubular shaped barrel filled with the liquid medicament 8. Towards the distal end the medicament container 5 comprises an outlet provided with a seal 7. The seal 7 is configured to become penetrated by a double-tipped injection needle (not shown). Towards the proximal direction 3 the barrel of the medicament container 5 is sealed by the stopper 6. The stopper 6 is displaceable towards the distal direction 2 through the action of the distally advancing piston rod 12. In this way a dose of the medicament 8 can be expelled from the medicament container 5. The medicament container 5 may be implemented as a standard cartridge for pen-type injection devices.

The distal housing component 40, hence the first housing component 40, may be implemented as a cartridge holder configured to accommodate and to hold the medicament container 5. The injection device 10 may be further provided with a protective cap 4. The protective cap 4 effectively covers at least a major portion of the distal end of the injection device 10. It may provide protection for the first housing component 14 and may enclose a major part of the first housing component 14.

The drive mechanism 11 comprises numerous mechanically engaging components, such as a dose button 14, a dose dial 16, and a number sleeve 17. The dose dial 16 may be rotationally supported on the second housing component 60. It may be dialed or rotated by a user for setting of a dose of variable size. A user may depress the dose button 14 at the proximal end of the injection device 10 for initiating or for controlling a dose dispensing or dose expelling action of the drive mechanism 11.

The number sleeve 17 is at least partially visible through a window 18 or aperture in the second housing component 60. The number sleeve 17 comprises a consecutive sequence of numbers being indicative of a size of a dose currently set by the drive mechanism 11. The drive mechanism 11 may be implemented all mechanically. for example, a force required for advancing the piston rod 12 in the distal direction 2 is either provided by a user depressing the dose button 14 in distal direction 2. Alternatively, the drive mechanism 11 is provided with a mechanical energy storage, such as a drive spring. Mechanical energy stored in the mechanical energy storage may be released upon depressing of the dose button 14. for example, the force required to advance the piston rod 12 in distal direction 2 is entirely or at least partially provided by the mechanical energy storage. Examples of drive mechanisms 11 to be implemented with the injection device 10 can be found in WO 2014/033195 A1 or WO 2014/033197 A1 the entirety of which being incorporated herein by reference. The drive mechanism as disclosed in WO 2014/033195 A1 is a reusable drive mechanism. The drive mechanism as disclosed in WO 2014/033197 A1 is an example of a disposable drive mechanism being void of a reset function.

With other examples the drive mechanism 11 can be implemented electromechanically. for example, an electromechanical actuator may provide the force or torque required for advancing the piston rod 12 in distal direction 2. As illustrated in greater detail in FIGS. 2-9 the first housing component 40 comprises a tubular-shaped sidewall 41 and a proximal connecting end 42 configured for attachment to the second housing component 60 or configured for attachment to an add-on device 100. Complementary, the second housing component 60 comprises a tubular-shaped sidewall 61 with a distal connecting end 62 configured for attachment to at least one of the proximal connecting end 42 and the add-on device 100.

The add-on device 100, which may be also denoted as an auxiliary device or electronic module, comprises a housing 101 and a mounting adapter 110. The mounting adapter 110 comprises a first connecting interface 140 and a second connecting interface 160. As illustrated in FIG. 2 the first connecting interface 140 forms or constitutes a distal connecting end of the mounting adapter 110. The first connecting interface 140 is configured for attachment and/or for fixing to the proximal connecting end 42 of the first housing component 14.

The add-on device 100, in particular its mounting adapter 110 further comprises a second connecting interface 160. The second connecting interface 160 is configured for attachment to the distal connecting end 62 of the second housing component 60. The mounting adapter 110 may comprise an adapter sleeve 112. The adapter sleeve 112 may comprise a somewhat tubular shape and may feature a cross-section or diameter that is equivalent or identical to the cross-section or diameter of at least one of the first and the second housing components 40, 60. The add-on device 100 further comprises an extension 114 connected to the adapter sleeve 112 by a narrowing or neck-shaped bridging portion 115. The bridging portion 115 protrudes radially outwardly from the adapter sleeve 112. The extension 114 may comprise rhombic shape and a display 218 and may extend along the longitudinal direction of the first and/or the second housing component 14, 16.

The add-on device 100 further comprises a fastener 102 for fastening the add-on device 100 and hence the mounting adapter 110 to at least one of the first housing component 40 and the second housing component 60. The combination of the add-on device 100 and the injection device 10 forms or constitutes a drug delivery system 1 as illustrated in FIG. 3.

In the example as illustrated in FIGS. 2-9 the proximal connecting end 42 of the first housing component 40 comprises a housing insert 44 configured for insertion into an interface receptacle 144 of the first connecting interface 140. The second connecting interface 160 comprises an interface insert 164 configured for insertion into a complementary shaped housing receptacle 64 of the second housing component 60.

Typically, the interface receptacle 144 is provided at a longitudinal distal end 142 of the first interface 140. The interface insert 164 is provided at a proximal longitudinal end 162 of the second connecting interface 160. The first connecting interface 140 comprises a tubular-shaped sidewall 141. The second connecting interface 160 comprises a tubular-shaped sidewall 161.

Typically, an outer cross-section or outside diameter of the housing insert 44 matches with an inside diameter or inner cross-section of the interface receptacle 144. Likewise, an outside diameter or outer cross-section of the interface insert 164 matches or corresponds with an inside diameter or inner cross-section of the housing receptacle 64. In this way a somewhat convoluted and nested engagement of the mounting adapter with the first and second connecting interfaces 140, 160 with complementary-shaped proximal and distal connecting ends 42, 62 of the first and second housing components 40, 60 can be provided. Such a nested or interleaved configuration provides a rather stable, long-lasting and robust mutual connection of the add-on device 100, the first housing component 40 and the second housing component 60.

The first housing component 40 may comprise a radially outwardly protruding flange 45 delimiting the longitudinal extent of the housing insert 44 in distal direction 2. In a final assembly configuration the proximally facing side of the flange 45 may abut with the longitudinal end 142 of the first connecting interface 140. The second connecting interface 160 may comprise a radially stepped portion 165 delimiting the longitudinal extent of the interface insert 164 towards the distal direction 2. In a final assembly configuration the stepped portion 165 is in longitudinal abutment with the distal end 62 or distal end face of the sidewall 61 of the second housing component 60. Moreover, a stepped portion 145 delimiting the interface receptacle 144 in longitudinal direction may be in abutment with a longitudinal end of a sidewall 41 of the first housing component 40.

As becomes apparent from the illustration of FIGS. 4 and 5 the outer diameter of the first housing component 40, in particular the outer diameter of the sidewall 41 of the housing insert 44 may be larger than the inner diameter of the housing receptacle 64 of the second housing component 60. With such a configuration the proximal connecting end 42 is incompatible with the distal connecting end 62. Consequently, the first housing component 40 cannot be fixed or connected directly to the second housing component 60. for example, use of the add-on device 100 and the mounting adapter 110 is indispensable for indirectly mutually connecting the first and second housing components 40, 60.

With such an example it is compulsory to make use of the add-on device 100 with its mounting adapter 110 to enable a mutual connection of the first and the second housing components 40, 60 via the add-on device.

With other examples the proximal connecting end 42 is compatible with the distal connecting end 62. Then, the first housing component 40 is directly connectable to the second housing component 60 and use of the add-on device 100 is only optional.

As it is further apparent from the illustration of FIGS. 5-8 the add-on device 100, in particular its mounting adapter 110 and the first and second housing components 40, 60 comprise mutually corresponding fastening elements. With the presently illustrated example the proximal connecting end 42 of the first housing component 40 is provided with a counter fastening element 46 implemented as a radial protrusion 48 on the sidewall 41 of the housing insert 44. On an inside of the sidewall 141 of the first connecting interface 140 there is provided a complementary shaped fastening element 146. for example, the fastening element 146 is implemented as a recess 148 complementary shaped to the protrusion 48.

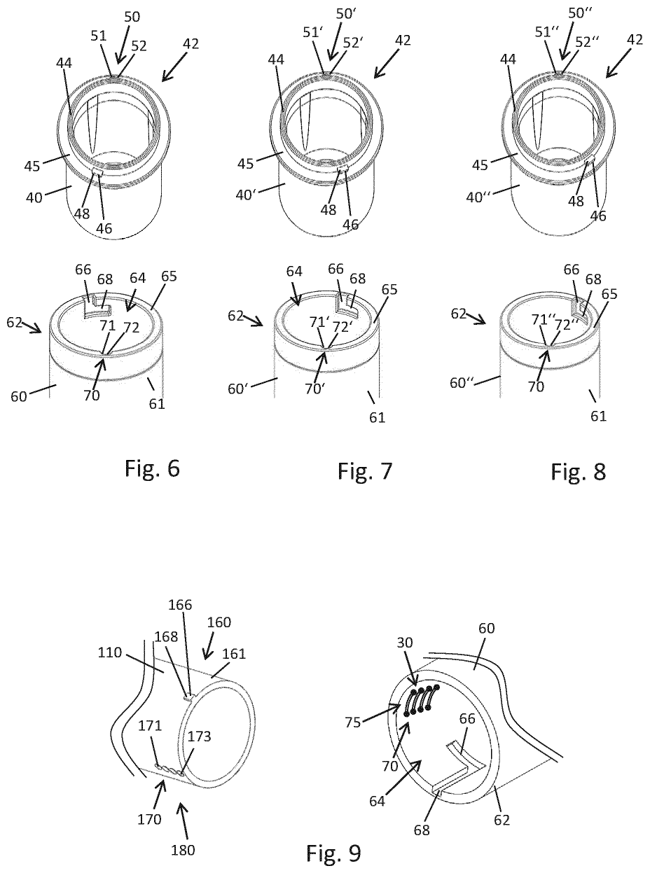

Correspondingly there is provided a second fastening element 166 on the interface insert 164 provided at the second connecting interface 160. The fastening element 166 is implemented as a radial protrusion 168 as shown in FIG. 9. The second housing component 60 comprises a complementary shaped outer fastening element 66 implemented as a radial recess 68 on an inside surface of the sidewall 61 of the second housing component 60. The fastening elements 146, 166 and the complementary-shaped counter fastening elements 46, 66 may provide a clip joint, a threaded joint or a bayonet as illustrated for instance in FIGS. 6-9.

The add-on device 100 may be used for a series or for different types of injection devices. Insofar, there may be provided a set or a kit of injection devices 10 that are substantially equivalent but distinguish by the amount or by the concentration of the medicament 8 as provided in the medicament container 5. To prevent unintended cross use of a first housing component of a first type of an injection device with a second housing component of a second, hence another type of an injection device 10′, the first housing component 40 and the second housing component 60 may be subject to a coding or counter coding.

The housing components 40, 60 of a first type of an injection device 10 are provided with a mutually corresponding coding and a counter coding so as to enable a mutual connection and fixing of the respective housing components 40, 60. Housing components 40′, 60′ of an injection device 10′ of a second time are provided with codings or counter codings of a different type so as to prevent unintended cross use.

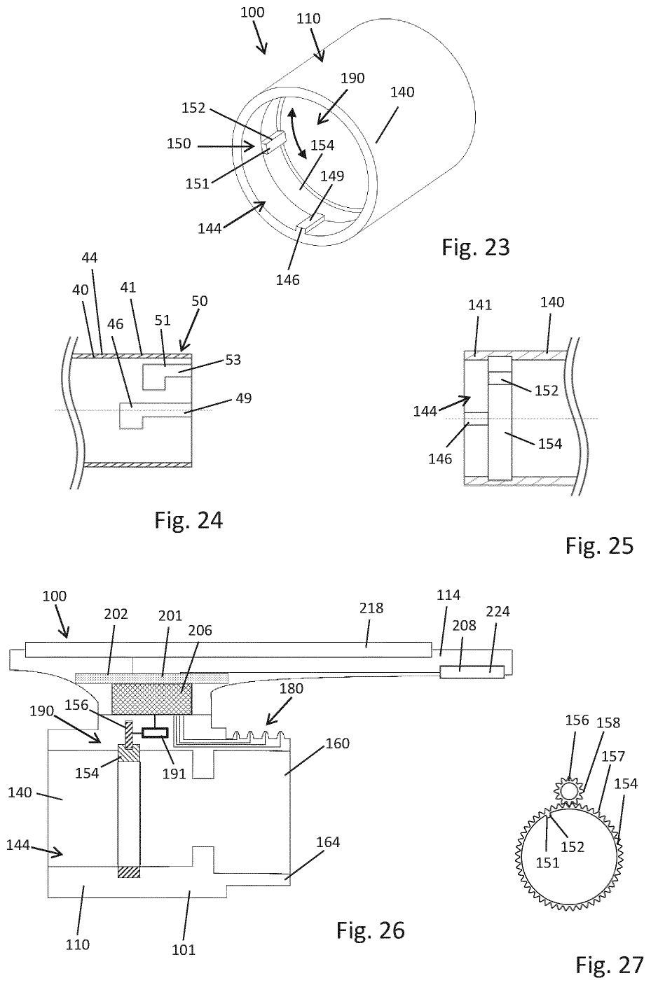

To enable and to support attachment of the add-on device 100 to the first and to the second housing components 40, 60 also the first connecting interface 140 is provided with a first coding 150, e.g., illustrated in FIG. 23. The second connecting interface 160 is provided with a second coding 170, e.g., illustrated in FIG. 9. In the examples of FIGS. 6-8 numerous examples of a coded implementation of first and second housing components 40, 50 is illustrated.

Here, the sidewall 41 of the first housing component 40 is provided with a first counter coding 50 complementary shaped to a second counter coding 70 as provided on the sidewall 61 of the second housing component 60. for example, the first counter coding 50 is provided with a first mechanical counter coding feature 51. The counter coding feature 51 comprises a counter coding protrusion 52 protruding radially outwardly from the sidewall 41 of the first housing component 40. The second counter coding 70 comprises a complementary shaped mechanical second counter coding feature 71 featuring a counter coding recess 72 as provided on an inside surface of the sidewall 61 of the second housing component. In FIG. 7 another type of a first and a second counter coding 50′, 70′ are illustrated. for example, the second counter coding 50′ also comprises a counter coding feature 51′ with a radially outwardly protruding second counter coding protrusion 52′. The second counter coding features 71′ comprises a counter coding recess 72′ complementary shaped to the first counter coding protrusion 52′. Compared to the first and second counter codings 50, 70 as illustrated in FIG. 6 the first and second counter coding 50′, 70′ as illustrated in FIG. 7 distinguish by the position of the counter coding features 51′, 71′ with respect to the position of the fastening elements 46, 66. This way and in an attempt to connect the first housing component 14 as illustrated in FIG. 6 with a second housing component 60′ as illustrated in FIG. 7 a mutual engagement of the fastening elements 46, 66 cannot be obtained because the first coding feature 51 will not be aligned with the second counter coding feature 71′ but will abut with the distally facing end face 65 of the sidewall 61 of the second housing component 60′.

In FIG. 8 a further example of a first and a second counter coding 50″, 70″ is illustrated. Also the first counter coding 50″ comprises a mechanical counter coding feature 51″ with a radially outwardly extending protrusion 52″. The first counter coding 50″ is arranged in relation to the fastening element 46 such that only a second housing 60″ as illustrated in FIG. 8 will be able to connect to the first housing component 40″. The second counter coding feature 71″ is located at a position relative to the fastening element 66 that matches the distance or relative position of the first counter coding feature 50″ with the fastening element 46.

The illustration and variation of numerous counter codings 50, 70, 50′, 70′, 50″, 70″ is only exemplary for a large variety of different mechanical codings. Typically, the first connecting interface 140 of the add-on device 100 may be provided with a coding 150 being somewhat identically shaped to the second counter coding 70 as provided on the second housing component 60. The second connecting interface 160 may be provided with a coding 170 that is somewhat identically shaped to the counter coding 50 as illustrated in any of the FIGS. 6-8.

In other words, the coding 150 as provided on the first connecting interface 150 may be shaped to complement the first counter coding 50 of the first housing component. The first coding 150 may comprise a first mechanical coding feature 151 comprising a complementary shaped radial recess (not illustrated). Likewise, also the second coding 170 as provided on the second connecting interface 160 may be provided with a mechanical coding feature 171 that is complementary shaped to the second counter coding feature 71 of the second housing component 60. Hence, the second coding feature 171 may comprise a radial protrusion (not illustrated) to engage with the counter coding recess 72 as provided on the second housing component 60.

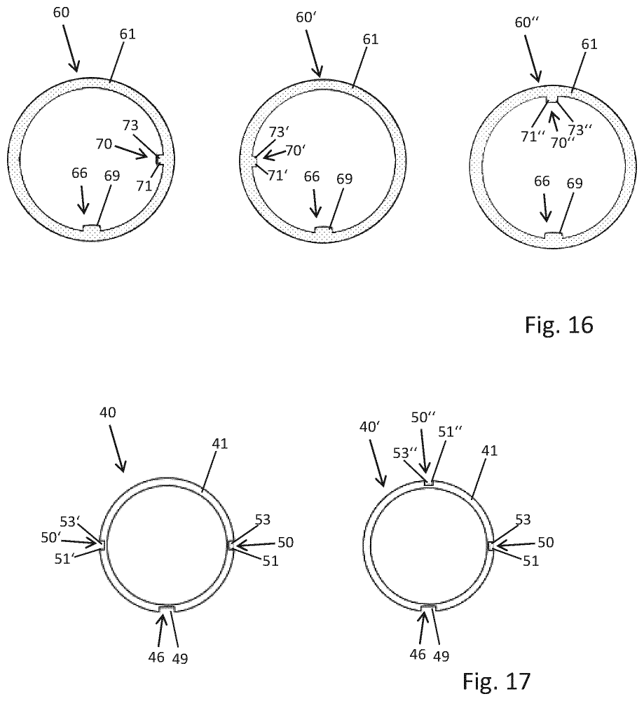

In FIGS. 16 and 17 another example of different types of counter codings is provided on the second housing component 60 and the first housing component 40 are illustrated. Again, the coding is defined by a tangential or circumferential position of the counter coding features 71, 71′, 71″ relative to a counter fastening element 66. With a first type of a second housing component 60 as illustrated in FIG. 16 the counter fastening element 66 comprises a radially inwardly extending protrusion 69 to engage with a complementary-shaped radial recess 46 as provided by the complementary-shaped counter fastening element 46 of the first housing component 40 as illustrated in FIG. 17.

The first housing component 40 comprises a housing insert 44 configured for insertion into the housing receptacle 64 of the second housing component 64. The first housing component 40 as illustrated in FIG. 17 is provided with a first counter coding 50 and with a second counter coding 50′. The first counter coding 50 and the second counter coding 50′ are provided on diametrically opposite sides on the outside surface of the sidewall 41. Both counter coding 50, 50′ comprise a counter coding recess 53, 53′, respectively. The counter coding recess 53 is complementary shaped to the counter coding protrusion 73 as provided on the inside surface of the sidewall 61 of the second housing component 60.

The second housing component 60 of a first type as illustrated in FIG. 16 is exclusively provided with only one counter coding 70. The second housing component 60′ of a second type is provided with a similar counter coding 70′ featuring a radially inwardly extending counter coding protrusion 73′ thus forming the respective counter coding feature 71′.

The counter coding 70′ of the second type is located at a different position compared to the counter coding 70 of the first type. In FIG. 16 a further type of a second housing component 60″ is illustrated. There, the respective counter coding 70″ is located at another circumferential position relative to the counter fastening element 66.

Complementary to the three different types of second housing component 60, 60′, 60″ there are provided only two first housing component 40, 40′ as illustrated in FIG. 17. The first housing component 40′ of the second type is provided with the first counter coding 50 and with a third counter coding 50″. The third counter coding 50″ distinguishes from the second counter coding 50′ by its circumferential position relative to the counter fastening element 46.

In effect the first housing component 40 of the first type can be combined and attached to the second housing component 60 of the first type or to the second housing component 60′ of the second type. It cannot be attached or connected to the second housing component 60″ of the third type because there, the first housing component is void of a counter coding to engage with the counter coding 70″ of the second housing component 60″ of the third type.

The first housing component 40′ of the second type as illustrated in FIG. 17 is connectable or engageable with the second housing component 60 of the first type and with the second housing component 60″ of the second type. It is not connectable to the second housing component 60′ of the second type.

The example of different counter codings 50, 50′, 50″, 70, 70′, 70″shows that the mutually corresponding codings and counter codings of the housing components 40, 60 may not only be restricted to enable connection of only one first housing component of a particular type with only one second housing component of the same type. Rather, the mutually corresponding codings and counter codings may also enable a connection of one coding of a first type with a coding of a first type and with another coding of another type.

The illustration of FIGS. 16 and 17 and the implementation of numerous counter codings with counter coding features is equally applicable to the respective codings 150, 170 as provided on the first connecting interface 140 and the second connecting interface 160 of the mounting adapter 110 or of the add-on device 100.

In the further example of FIG. 9 the second connecting interface 160 is provided with a second coding 170 complementary-shaped to a second counter coding 70 as provided on an inside surface of the sidewall 61 of the second housing component 60. The counter coding 70 as provided on the sidewall 61 of the second housing component 60 may represent a code 30 to distinguish the respective housing component 60 with its drive mechanism 11 from another type of a housing component 60′ with a differently configured drive mechanism 11′ (not illustrated). The code 30 or the counter coding 70 may be implemented as an electrical or optical counter coding feature. The complementary shaped coding 170 may provide an electrical coding but may also comprise or may belong to a code recognition unit 180 operable to read or to detect the electrically or optically implemented counter coding feature 75. With some examples the coding 170 may coincide with the code recognition unit 180.

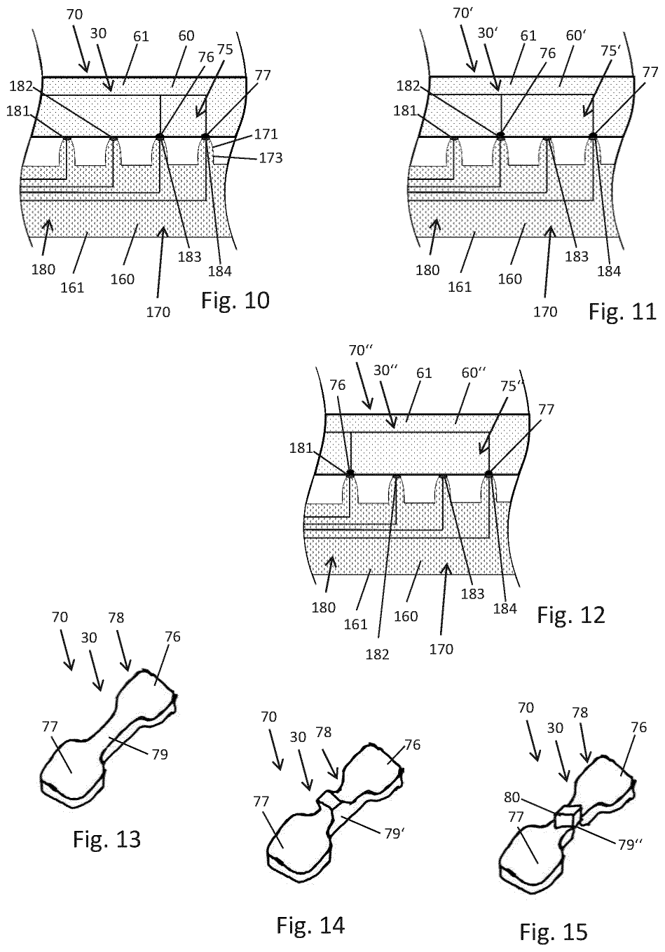

With other examples the coding 170 may comprise a mechanical coding feature 171, e.g., protruding from a sidewall 161 and forming or constituting at least one coding protrusion 173, e.g., indicated in FIG. 10.

In the example of FIGS. 10-15 the second counter coding 70 as provided on the second housing component 60 comprises an electric counter coding feature 75. The electric counter coding feature 75 represents a code 30 to distinguish the second housing component 60 and/or its drive mechanism 11 from another housing component 60′. The second connecting interface 160 of the add-on device 100 comprises a coding 170 to cooperate with the counter coding 70. The second coding 170 comprises a code recognition unit 180 operable to detect an/or to capture the code 30 or the counter coding 70 as provided on the distal connecting end 62 of the second housing component 60.

The counter coding 70 comprises two electrical contacts 76, 77 that make electrical contact with two complementary-shaped electrical contacts 181, 182, 183, 184 of the code recognition unit 180. The electrical contacts 181, 182, 183, 184 as provided on the second connecting interface 160 are separated in longitudinal direction. They are provided at discrete and well-defined positions on the outside surface of the sidewall 161 of the second connecting interface 160.

With the example of a counter coding 70 of a first type as illustrated in FIG. 10 the electrical contacts 76, 77 of the counter coding 70 are in contact with the two electrical contacts 183, 184 of the code recognition unit 180. A different type of a counter coding 70′ and hence a different type of an electrically implemented code 30′ is shown in FIG. 11. There, the position of the electrical contact 76 has changed compared to the counter coding 70 of FIG. 10. The electrical contacts 77 is in contact with the electrical contact 184 but the electrical contact is 76 is now in electrical contact with the electrical contact 182. The code recognition unit 180 may then sense, that only the electrical contacts 182 and 184 are in electrical contact with the complementary shaped electrical contacts 76, 77 of the counter coding 70′.

In still another example of a further counter coding 70″ and hence of another electrically implemented code 30″ the electrical counter coding feature 75″ also comprises two electrical contacts 77, 76 but here and contrary to the configuration of the electrical counter coding features 75, 75′ as illustrated in FIGS. 10 and 11 the electrical contact 76 of the electrical counter coding feature 75″ is in contact with the electrical contact 181 of the code recognition unit 184.

The different configurations, e.g., the positions of the electrical contacts 76, 77 on the sidewall 61 of the second housing component 60 enabled to distinguish between differently encoded housing components 60, 60′, 60″. With the electric detection of a code 30, 30′, 30″ and hence with the electric detection of the counter coding 70, 70′, 70″ one of the electrical contacts 76, 77 and the electrical contacts 181, 182, 183, 184 comprises a protruding electrical contact pin whereas the other one of the electrical contacts 76, 77 and the electrical contacts 181, 182, 183, 184 comprises a planar-shaped contact pad, e.g., flush mounted in an inside or outside surface of the sidewall 161, 61 of the mounting adapter 110 or of the second housing component 60.

The individual electrical contacts 181, 182, 183, 184 of the code recognition unit 180 may be individually connected to a processor 202 or signal processing unit of the add-on device 100. The electrical contacts 76, 77 of the counter coding 70 or of the code 30 may be electrically connected to each other. In this way, the counter coding 70 cooperating with the code recognition unit 180 may provide an electrical contact between two of the electrical contacts 181, 182, 183, 184 of the code recognition unit 180.

In the example of FIG. 10, there is provided an electrical contact between the electrical contacts 183, 184. With the example of FIG. 11, the electrical contact 182 is electrically connected to the electrical contact 184. With the example of FIG. 12 the electrical contact 181 is electrically connected to the electrical contact 184. The opposite ends of the electrical contacts 181, 182, 183, 184 (not illustrated) may be individually connected to the processor 202 and a binary coding can be provided.

With FIG. 13, another example of a counter coding 70 or code 30 featuring an electric coding feature is illustrated. The counter coding 70 or the code 30 comprises an electrical resistor 78 comprising a first electrical contact 76 and a second electrical contact 77. The resistor 78 comprises an electrically conductive material. The oppositely located ends of the resistor 78, each of which provided with an electrical contact 76, 77 are connected by a neck or bridging portion 79. Depending on the choice of material or depending on the geometry of the electrical contacts 76, 77 and/or depending on the geometry of the neck portion 79, the electric resistance between the electric contact 76 and the electric contact 77 may vary. The code recognition unit 180 may comprise a first electrical contact 181 connectable to the electrical contact 77 and a further electrical contact 182 connected to the further electrical contact 76. Now, the code recognition unit 180 may be configured to measure the magnitude of the electrical resistivity between the first electrical contact 181 and the second electrical contact 182, which may vary depending on the choice or configuration of the resistor 78, which is a fixed and hence unmodifiable resistor.

Here, the magnitude of the electrical resistance measurable between the electrical contacts 76, 77 may constitute the electrical coding for the respective housing components 40, 60.

With the further example of a resistor 78 as illustrated in FIG. 14 the neck portion 79′ may have a different design compared to the neck portion 79 of the resistor 78 as illustrated in FIG. 13. The geometric design or the choice of a material of the neck portion 79 may provide a variation of the electrical resistance between the electrical contacts 76, 77. The neck portion 79′ may vary in thickness or cross-section in order to modify the electrical resistance of the resistor 78.

With the further example of another resistor 78 as illustrated in FIG. 15 there may be provided a surface mounted resistor 80 in the neck portion of 79″ between the electrical contacts 76, 77. For varying the electrical resistivity between the electrical contacts 76, 77 a surface mounted resistor 80 of appropriate resistivity may be selected and mounted on the neck portion 79″. Instead of a surface mounted resistor 80 there may be also used a surface mounted coil. Accordingly, the code recognition unit 180 may then be configured to measure a size or magnitude of an inductivity provided by the surface mounted coil.

With another example (not illustrated) the electric counter coding feature 75 can be provided on an outside surface of one of the first and the second housing components 40, 60 while the code recognition unit 180 is provided on an inside surface of the housing 101 of the add-on device 100.

Attachment of the electrically implemented counter coding features 75 to any of the housing components 40, 60 can be provided e.g., by an adhesive foil provided, e.g., printed with electrically conductive conductor paths with an electrically conductive structure.

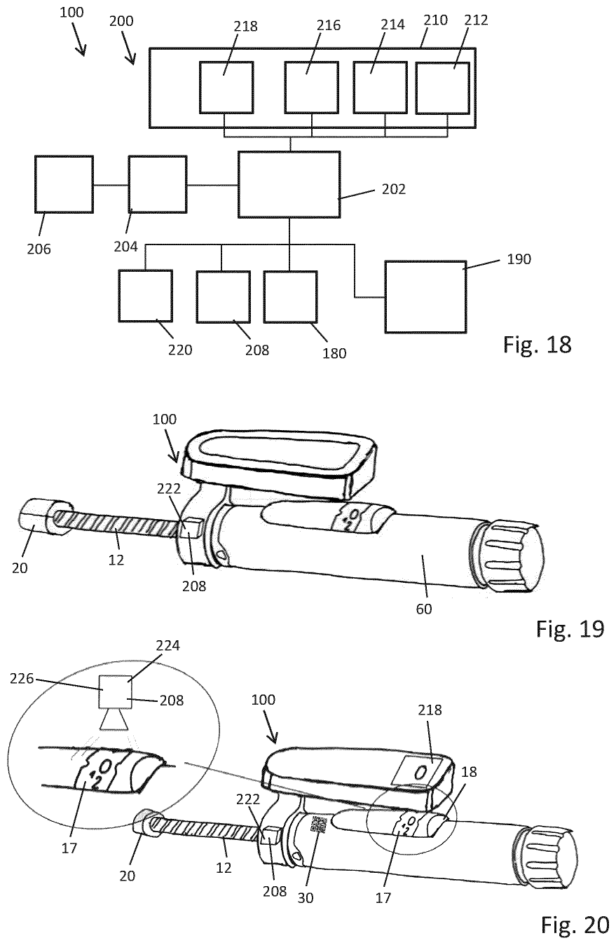

In FIG. 18 a block diagram of an exemplary implementation of the add-on device 100 is schematically illustrated. The add-on device 100 comprises a logic circuit 200, which may be provided on a printed circuit board 201, e.g., illustrated in one of FIG. 27, 29 or 34. The logic circuit 200 may comprise a processor 202, a communication unit 210 as well as a sensor 208. The logic circuit 200 may comprise a switch 204 by way of which the processor 202 is connected to an electric energy storage, e.g., implemented as a battery 206. The processor 202 is further connected to a memory 220, to an optional code recognition unit 180 as well as to an optional coding configurator 190, the function of which will be explained below.

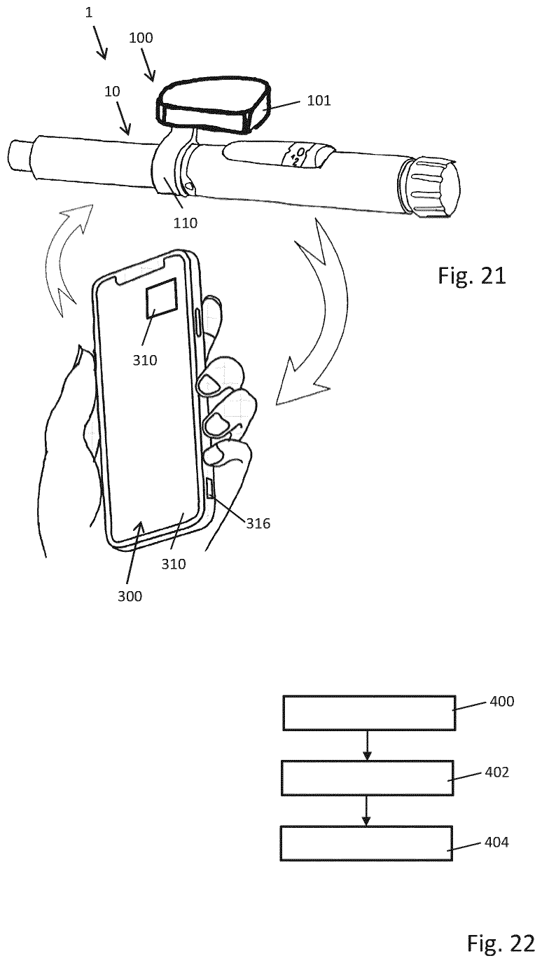

The communication interface 210 may comprise at least one of a display 218, a speaker 216 and a transceiver 214. The transceiver 214 is particularly adapted to establish a communication link with an external electronic device 300 as illustrated in FIG. 21. The communication link may be based on radio frequency electromagnetic signals, it may be implemented on a standard communication protocol. The communication link may be realized by one of a Wi-Fi connection, a Bluetooth connection, a NFC connection, an RFID connection and the like. The communication interface 210 may also comprise an input 212 by way of which a user is given the possibility to modify or to alter operation of the add-on device 100.

The external electronic device 300 comprises a communication interface 310 in order to establish a communication link with the transceiver 214 of the communication unit 210. Typically, the external electronic device 300 comprises at least a display 318. The external electronic device 300 may optionally also comprise a speaker 316. With some implementations, e.g., as illustrated in FIG. 21, the add-on device 100 may be void of an own display 218 and/or void of an own speaker 216.

Here, user interaction may take place the via the transceiver 214 and the portable external electronic device, via the wireless communication between the communication interfaces 210, 310.

The add-on device 100 is particularly adapted and implemented to assist a user in using or operating the injection device 10. For this, the add-on device 100 may be configured to provide visual or audible information to the user via the display 218, 318 and/or via the speaker 216, 316. The information provided by the add-on device 100 may be selected in accordance to a code 30 or counter coding 50, 70 recognized by the code recognition unit 180, e.g., in the course of assembling the add-on device 100 to at least one of the first and second housing components 40, 60.

By recognizing or detecting the code 30 or the counter coding 50, 70 the add-on device 100 is inherently provided with information related to the specific type of the housing component 40, 60 or injection device 10. Injection devices 10 of different type may require providing of different device specific information to a user. By automatically detecting the type of injection device via the code recognition unit 180 the processor 202 is operable to select and to provide the correct information that correlates with the injection device 10 actually in use.

Additionally or alternatively, the add-on device 100 is operable to monitor repeated use or operation of the injection device 10. For this, the add-on device 100 is equipped with at least one sensor 208 or a sensing unit. The sensor 208 is operable to detect a user-induced operation of the injection device 10, e.g., during setting of a dose and/or during dispensing of the dose. The logic circuit 200 and/or the processor 202 is provided with a clock by way of which a point in time at which a particular device operation takes place can be recorded and logged.

Any operation of the injection device 10, e.g., any dose setting and/or any dose dispensing can be assigned with a timestamp and can be stored in the memory 220. This way a dosing history can be recorded.

In FIG. 19 one example of a sensor 208 is illustrated. The sensor 280 is implemented as a magnetic sensor 222 located inside or on the housing 101 of the add-on device 100. The magnetic sensor 222 may cooperate with at least one of the piston rod 12 and the pressure piece 20 of the piston rod 12. At least one of the piston rod 12 and the pressure piece 20 is provided with a permanent magnetic encoding. When the piston rod 12 is subject to a distally directed displacement relative to the magnetic sensor 222 a respective movement can not only be detected but can also be quantitatively determined with the magnetic sensor 222. Likewise, also the number sleeve 17 or any other movable component of the drive mechanism 11 indicative of the size of a dose may be magnetically encoded so as to enable a precise measurement of the movement of such a component by the magnetic sensor 222.

The magnetic sensor 222 may operate like a metal detector. It may track or detect smallest movements of the piston rod 12. The piston rod 12 may be made of a metal, it may comprise a magnetized plastic or a metal coated plastic material. The magnetic sensor 222 may be implemented as a hall sensor or as an AMR sensor.

With the further example of FIG. 20 the sensor 208 is implemented as an optical sensor 224. The sensor 208 may be provided on a radially inwardly facing side of the extension 114 of the add-on device 100. The sensor 208 and hence the optical sensor 224 may comprise a camera 226 to capture or read the numbers as provided on the number sleeve 17 that show up in the window 18 of the second housing component 60. The camera 226 or the optical sensor 224 may be provided with an optical character recognition unit so as to provide information about the dose size to the processor 202.

The sensor 208 or sensor arrangement may comprise a combination of a magnetic sensor 222 and an optical sensor 224. In this way, a redundant dose detection can be provided and the measurement signals of e.g., the magnetic sensor 222 can be compared with measurement signals or data obtained from the optical sensor 224

In the flowchart of FIG. 22 an example of a method of configuring the add-on device 100 is schematically illustrated. In a first step 400 the add-on device 100 is attached to the device housing 22 of the injection device 10. In step 402 the at least one code 30 or the counter coding 50, 70 as provided on the device housing 22 or as provided on one of the housing components 30, 60 is detected by the code recognition unit 180. Depending on the detected code in step 404 at least one of an execution and a calibration of at least one electronically implemented function of the device is modified on the basis of the detected or captured code 30 or counter coding 50, 70 of the injection device 10.

In this way, the add-on device 100 can be electronically adapted to the specific type of injection device 10. This enables to provide an add-on device 100 universally usable for a large variety of different types of injection devices 10. Upon mounting or attaching the add-on device 100 to the injection device 10 a code 30 or counter coding 50, 70 being indicative of the specific type of the injection device is automatically detected or captured.

Based on the detected or captured code, an injection device specific setting of the add-on device can be conducted or executed. For instance, with an injection device 10 of a first type a distally directed advancing motion of the piston rod 12 detected by the detector 208 is representative of a first size of a dose. With an injection device 10′ of a second time the same advancing motion of the piston rod 12 corresponds to a second dose size different from the first dose size. By automatically detecting the type of injection device 10, 10′ upon mounting or attaching the add-on device 100 to the injection device 10, 10′ respective settings of the add-on device 100, such as a calibration of a sensor 208 can be automatically provided.

Alternatively or additionally detection of a specific type of an injection device 10, 10′ by the add-on device 100 may invoke selection and presentation of different and hence injection device specific information to the user, via the communication interface 210.

With typical examples, the code 30 or the counter coding 50, 70 as provided on the injection device 10 is indicative of at least one of a type of the medicament, a concentration of a pharmaceutical active substance of the medicament, a size of the medicament container, a type of the drive mechanism, a manufacturing date of the medicament and an expiry date of the medicament.

With some examples the code 30 may be printed on the outside surface of one of the first and second housing components 40, 70. The code 30 or the counter coding 50, 70 may comprise a one or two-dimensional visual code, e.g., a data matrix or a QR code that is detectable e.g., by the optical sensor 224 of the add-on device 100.

With the present terminology a code 30 is provided on an outside surface of the device housing or inside the device housing 22, whereas the counter coding 50, 70 is provided on one of the connecting end 42, 62 of the first housing component 40 and the second housing component and 60, respectively. Both, the code 30 and the counter coding 50, 70 are detectable and/or capturable by the add-on device in order to distinguish the type of injection device from other types of injection devices.

Generally, the code 30 and the code recognition unit 180 can be implemented in a number of different ways. With one example the code 30 or the counter coding 50, 70 can be implemented as a mechanical code. The code recognition unit 118 may comprise a mechanical switch to mechanically detect the presence or configuration of the mechanically implemented code 30 or counter coding 50, 70.

With other examples the code 30 and/or the counter coding 50, 70 is implemented as an electric code. The code recognition unit 180 can be implemented to measure at least one electrical property of the code when in electrical contact with the electric code 30 or counter coding 50, 70.

With further examples the code 30 and/or the counter coding 50, 70 is implemented as a magnetic code. For example, the code recognition unit 180 comprises a magnetic sensor, such as a Hall sensor, an AMR sensor or a Wiegand sensor.

With further examples, the code 30 and/or the counter coding 50, 70 is implemented as a visual or optical code. It may comprise a QR code or a data matrix code readable or detectable by the code recognition unit 180, e.g., comprising at least one of an optical sensor 224 and a camera 226. The code recognition unit 180 may be implemented in the sensor 208 of the add-on device 100.

With further examples the optical sensor 224 or the camera 226 of the sensor 208 may be operable to conduct a contour comparison or contour check of dedicated contours or shapes of dedicated portions of the drug delivery device 10.

With further examples, the code 30 may be implemented electronically, e.g., by an electronic chip provided on or embedded in the housing 22 of the injection device 10. The electronic chip may enable wireless or wired connected communication with the logic circuit 200 so as to identify the electronic chip and hence the respective injection device 10.

In FIG. 23 a reconfigurable mechanical coding 150 is provided on or in the first connecting interface 140 of the add-on device 100. The first connecting interface 140 comprises an interface receptacle 144 shaped and configured to receive a complementary housing insert 44 of the first housing component 40.

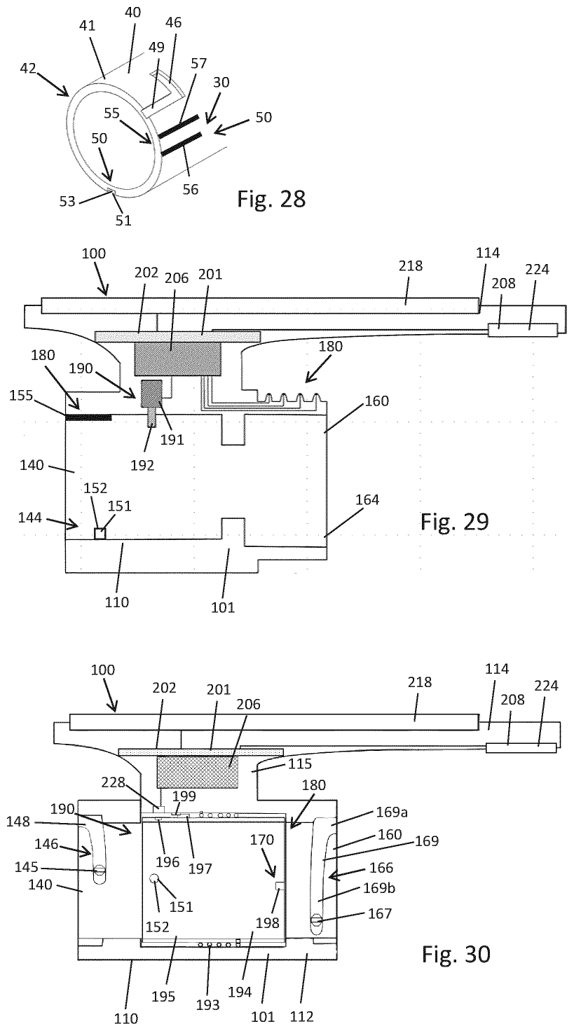

As described before the inside surface of the sidewall 141 of the first connecting interface 140 comprises a fastening element 146. The fastening element 146 comprises a radially inwardly extending protrusion 149 to engage with a complementary shaped recess 49 on an outside surface of the sidewall 41 of the first housing component 40, e.g., as shown in FIG. 28. As illustrated in FIG. 24 the counter fastening element 46 comprises a L-shaped recess or L-shaped fastening groove. Hence, there is provided a bayonet joint for connecting the first connecting interface 140 and the proximal connecting end 42 of the first housing component 40.

In addition, there is provided a counter coding 50 on the sidewall 41 of the first housing component 40. The counter coding 50 comprises a counter coding recess 53. The counter coding recess 53 is provided at a dedicated tangential or circumferential offset from the counter fastening element 46 as it is immediately apparent by the perspective illustration of FIG. 28.

The counter coding 50 and hence the counter coding feature 51 is defined by the circumferential distance or tangential distance between the counter coding recess 53 and the counter fastening element 46.

The reconfigurable coding 150 comprises a coding protrusion 151 protruding radially outwardly from the sidewall 141 of the first connecting interface 140. As it is further apparent from the sketches of FIGS. 23-27 the coding protrusion 152 is provided on a ring 154 rotationally supported on or inside the sidewall 141. In this way, and by moving or rotating the ring 154 the position of the coding feature 151 and hence of the coding protrusion 152 relative to the fastening element 146 can be modified. For moving or rotating the ring 154 the add-on device 100 comprises an electromechanical actuator 191, e.g., implemented as a motor or stepper motor.

The motor or drive 191 is connected or engaged with a gear wheel 156. The gear wheel 156 comprises teeth 158 that mesh with teeth 157 on the outside surface of the ring 154. This way and by activating the actuator 191, e.g., by the processor 202, the ring 154 can be rotated relative to the housing 101 of the add-on device 100. In this way the position, in particular the circumferential or tangential position of the coding protrusion 152 and hence of the coding feature 151 can be modified.

The reconfigurable or modifiable mechanical coding 150 is operably connected with a coding configurator 190. The coding configurator comprises the actuator 191, the gear wheel 156 and the ring 154. In this way and when a particular type of a second housing component 60 has been detected or recognized on the second connecting interface 160, e.g., by the code recognition unit 180, the add-on device 100, in particular the processor 202 may be operable to control or to initiate a reconfiguration of the first coding 150 as provided on the first connecting interface 140. Typically, the first coding 150 is reconfigurable by the coding configurator 190 so as to provide a first coding 150 that prevents attachment and/or connection of an unsuitable first housing component 40 to the add-on device 100.

In effect, a code 30 or a second counter coding 70 as provided on the second housing component 60 can be detected by the code recognition unit 180. In response to the detection of the code 30 or of the counter coding 70 the coding configurator 190 is operable or actually operated to modify or to reconfigure the first coding 150 such that only a dedicated first housing component 40 intended for use with the second housing component 60 can be connected to the connecting interface 140 of the add-on device 100.

With the further example of FIGS. 28 and 29 the first counter coding 50 comprises a first counter coding feature 51 implemented as a mechanical counter coding feature and a second counter coding feature 55 implemented as an electric counter coding feature. The mechanical counter coding feature 51 comprises a counter coding recess 53 in the sidewall 41. The electric counter coding feature 55 comprises a first conducting path 56 and a second conducting path 57.

The first connecting interface 140 of the add-on device 100 as illustrated in FIG. 29 comprises at least one of an electric coding 155 and a mechanical coding feature 151. The coding feature 151 comprises a coding protrusion 152 complementary shaped to the counter coding recess 53.

The electric coding 155 may comprise a transverse or tangentially extending conductive structure, e.g., an electrical contact 155 connected to the processor 202. By way of the electric coding 155 cooperating with the electric counter coding feature 55 an electronic code detection can be provided similar to the code recognition as described above with the code recognition unit 180. Moreover, the electric coding 155 may belong to the code recognition unit 180 or may form part of another code recognition unit 180 particularly adapted to electrically detect or to capture at least one code 30 or counter coding 50, 70 provided on one of the first housing component 40 and the second housing component 60.

The electric or electronic counter coding feature 55 provides the benefit, that e.g., selection of a particular first housing component 40 of a number of available housing components 40′, 40″ can be reconfigured by a reprogramming or by reconfiguring of the add-on device 100. At a particular time, the add-on device 100 may be configured to allow connection to a first housing component 40 of a first type equipped with a first type of electronic counter coding feature 55 and to disable a connection of a first housing component 40′ of a second type equipped with another type of an electronic counter coding feature 55′. By reconfiguring or reprogramming the add-on device 100 the situation may change. Then, the first housing component 40 of the first type identified by the electronic counter coding feature 55 may become disabled while a first housing component 40′ of a second type identified through the electronic counter coding feature 55′ may become enabled for use with the add-on device 100.

With the example of FIG. 29 the coding configurator 190 comprises an actuator 191 operably engaged with a latch 192. By activating the actuator 191 the latch 192 is movable in radial direction so as to enable or to disable insertion of the first housing component 40 into the interface receptacle 144. With some examples the latch 192 may be implemented as a movable bolt. It may provide or contribute to the fastening element 146 configured for detachably fastening the first housing component 40 to the first connecting interface 140.

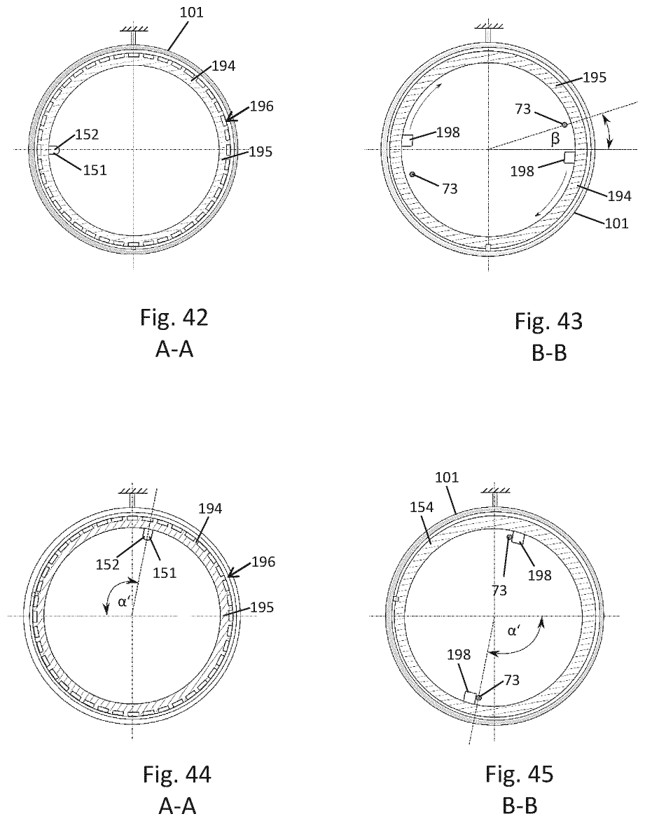

With the further example of FIGS. 30-45 the coding configurator 190 is implemented all mechanically. The coding configurator 190 comprises a follower 194 comprising a follower sleeve 195. The follower 194 and hence the follower sleeve 105 are rotationally supported inside the housing 101 of the add-on device 100. The follower 194 and hence the follower sleeve 195 is rotationally supported in or on the add-on device 100 against the action of a return spring 193. The return spring 193 is one of a torsion spring and a helical spring. It may extend around a portion of the outer circumference of the follower sleeve 195. One end of the spring is engaged or connected to the follower sleeve 195. Another end of the spring 193 is connected or engaged with the housing 101.

On an outside surface of the follower sleeve 195 there is provided an end stop 197 configured to engage with a complementary shaped stop 199 of the housing 101. When the stop 197 is in tangential or circumferential abutment with the stop 199 the follower sleeve 195 is in an initial configuration. It is kept in this initial configuration by the action of the return spring 193. As further indicated in FIG. 30 the first connecting interface 140 is provided with a first connecting element 146 implemented as a L-shaped recess 148. At or near the dead end of the recess 148 there may be provided a snap feature or snap element 147 to form a snap fit engagement with a complementary shaped protrusion 48 as provided on the sidewall 41 of the first housing component 40.

At the oppositely located second connecting interface 160 there is provided a second fastening element 166. The second fastening element 166 is implemented as a recess 169. The recess 169 is also of L-shape and provides a bayonet joint when engaged with a complementary shaped counter fastening element 66 of the second housing component 60. Also, the dead end of the L-shaped groove or recess 169 is provided with a snap element 167.

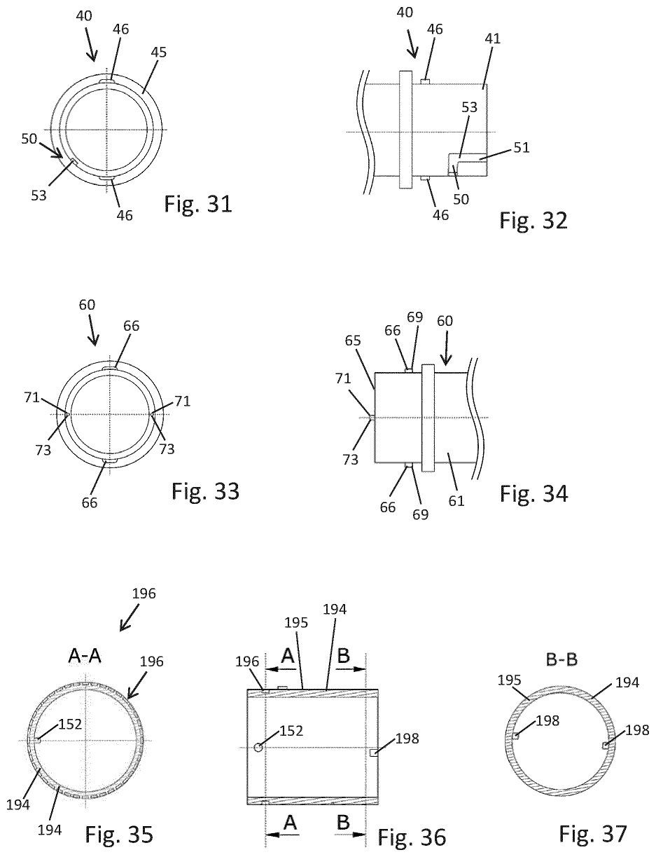

The L-shaped recess 169 comprises a first groove portion 169a and a second groove portion 169b. The first groove portion 169a extends in longitudinal direction and merges into the second groove portion 169b extending in circumferential or tangential direction. In FIGS. 31 and 32 a first housing component 40 connectable to the first connecting end 140 of the add-on device 100 of FIG. 30 is illustrated. In FIGS. 33 and 34 a second housing component 60 configured for attachment and fixing to the second connecting interface 160 is shown.

The first housing component 40 comprises a counter coding 50 defined by the position of a counter coding recess 53 relative to the counter fastening element 46. The counter coding recess 53 is configured to engage with a complementary-shaped coding protrusion 152 as provided at the distal end of the follower sleeve 195 as shown in FIGS. 35 and 36.

The second housing component 60 comprises counter fastening element 66 implemented as radial protrusion 69 at a longitudinal end face 65 of the sidewall 61 of the second housing component 60. There is provided a counter coding 70 comprising a mechanical counter coding feature 71 comprising longitudinally extending a counter coding protrusion 73. The counter coding protrusion 73 protrudes from the end face 65 of the sidewall 61. The counter coding protrusions 73 are configured to engage with at least one or with two complementary-shaped abutments 198 provided at or near the proximal end of the follower sleeve 195 as illustrated in FIGS. 36, 37 and 39.

The tangential position or circumferential position of the counter coding features 71 and hence of the counter coding protrusions 73 relative to the counter fastening element 66 defines the mechanical counter coding 70 of the second housing component 60.

With the present example it is intended that the distal connecting end 62 of the second housing component 60 is inserted into an interface receptacle 164 provided at the proximal end of the mounting adapter 110. Then, with the mechanical counter coding 73 as shown in FIG. 33 the counter coding protrusions 73 get in direct circumferential or tangential abutment with the radially inwardly extending abutments 198 provided on the inside of the follower 194 and protruding radially inwardly from a sidewall of the follower sleeve 195.

In the course of assembly the second housing component 60 to the second connecting interface 160 and hence in the course of moving the counter fastening element 60 along the second groove portion 169b the counter coding protrusions 73 induce a torque onto the abutments 198 thereby inducing a rotation of the follower sleeve 195 against the force provided by the return spring 193. When reaching a final assembly configuration, in which, e.g., the counter fastening element 66 engages with the snap element 167 the second housing component 60 is kept and fixed in a final assembly position on or in the add-on device 100.

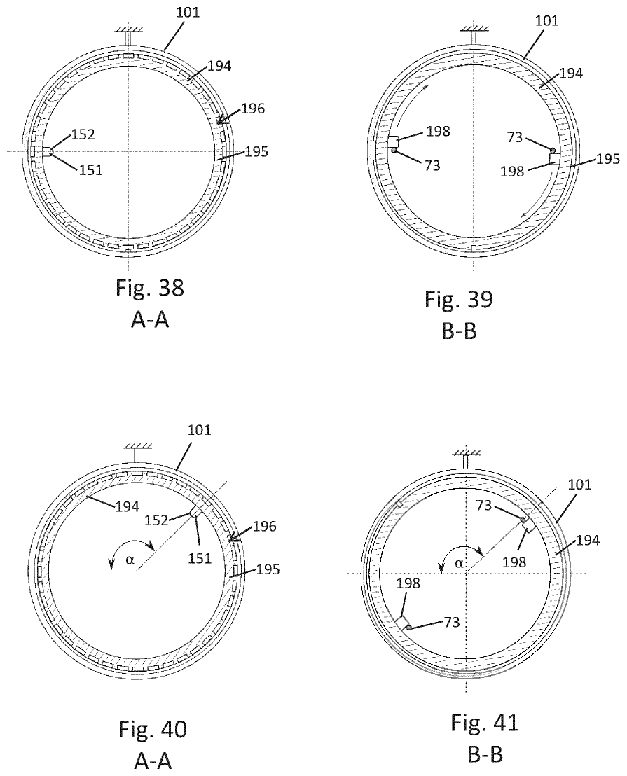

The rotation of the follower sleeve 194 as induced by the counter coding features 71 of the second housing component 60 induces a rotation of the coding feature 151 and hence of the coding protrusion 152 at the distal end of the follower sleeve 195 by an angle α as it is immediately apparent from a comparison of FIGS. 38 and 44. Now, and when making use of a different type of a counter coding 70 as provided on a second type of a second housing component 60′ as for instance illustrated in FIG. 43 the counter coding features 71′ and hence the counter coding protrusion 73′ are located at an angular offset α compared to the counter coding feature 71 and counter coding protrusion 73 of the example of FIG. 39 or 41.

When the second housing component 60 is subject to a rotation thereby following the contour of the fastening element 166 the second housing component is subject of a rotation of an angle β before the counter coding protrusions 73′ engage the abutments 198 of the follower sleeve 195. Since the counter fastening element 66 of the differently configured second housing components 60, 60′ remains unaltered, the total angle of rotation for reaching the final assembly configuration remains unaltered compared to the example as described in connection with FIGS. 38-41.

Insofar, and when reaching a final assembly configuration as illustrated in FIGS. 44, 45 the coding feature 151 and hence the coding protrusion 152 has rotated by an angle α′, which is smaller than the angle α as shown in FIGS. 40, 41. Here, α′=α−β.

Accordingly and with the modified counter coding 70′ and the modified position of the counter coding features 71′ the first coding feature 151 and hence the respective coding protrusions 152 configured to engage with the counter coding 50 of the first housing component will be located at a different position relative to the first fastening element 146. In this way, a mechanically implemented coding configurator 190 is provided.

Here, a mechanical counter coding 70 as provided at the distal connecting end of the second housing component 60 is operable to modify or to reconfigure the first coding 150 as provided on the first connecting interface 140. In this way, it can be provided that only one or a few of a number of available first housing components 40, 40′ can be connected to the first connecting interface 140 of the add-on device 100 when the second connecting interface 160 is connected to one of a plurality of available second housing components 60, 60′. This has the benefit that one and the same add-on device 100 can be used with a variety of differently encoded or coded housing components 40, 60.

The add-on device 100 can be connected to the second housing component 60 thereby modifying the first coding 150 of the add-on device 100 so that only one or a few dedicated first housing component 40 intended for connection or for cooperation with the selected second housing component 60 can be connected to the add-on device 100. Unintended cross use of a first housing component 40 not configured for use with a second housing component 60 can be effectively prevented.

In order to enable an electronic adjustment or modification of at least one electronically implemented function of the add-on device 100 the follower sleeve 195 is provided with a rotary encoding 196, e.g., provided on an outside surface of the follower sleeve 195.

The rotary encoding 196 may comprise a binary code or a gray code detectable by a rotary sensor 228 provided in or on the add-on device 100. This way, the degree of rotation and/or the magnitude of a movement of the follower sleeve 195 relative to the housing 101 in the course of attaching the second housing component 60 to the add-on device 100 can be measured and/or monitored. The rotary sensor 228 is operable to generate an identification signal by way of which the processor 202 is able to distinguish between counter coding 70, 70′ of different type Depending on the measured degree of rotation the logic circuit 200 and/or the processor 202 is provided with unequivocal information about the specific type of the second housing component 60.

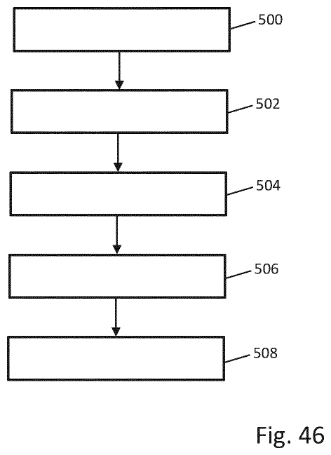

In the flowchart of FIG. 46 another method of using the add-on device 100 is described. In a first step 500 a housing component, e.g., the second housing component 60 is connected or attached to the add-on device 100. In the course of fastening the second housing component 60 to the add-on device 100 in step 502 the second housing component 60 is rotated relative to the housing 101 of the add-on device 100. In the course of the rotation conducted in step 502 and until the final assembly configuration of the first housing component 60 and the add-on device 100 has been reached the distal code and hence the reconfigurable code 150 of the add-on device 100 is modified or reconfigured in step 504.

In a subsequent step 506, the degree of rotation of the follower sleeve 195 and hence the magnitude of a rotation angle of the follower sleeve rotated in the course of attaching and fastening the second housing components to the add-on device 100 is detected or determined. According to the detected degree of rotation, in step 508 the add-on device is reconfigured or calibrated. At least one electronically implemented function of the add-on device 100 for assisting a user in operating the injection device 10 and/or for monitoring operation of the injection device 10 is adapted in accordance to the detected code 30 or counter coding 70.

### REFERENCE NUMBERS

1 drug delivery system

2 distal direction

3 proximal direction

4 protective cap

5 medicament container

6 stopper

7 seal

8 medicament

10 injection device

11 drive mechanism

12 piston rod

14 dose button

16 dose dial

17 number sleeve

18 window

20 pressure piece

22 device housing

30 code

40 housing component

41 sidewall

42 proximal end

44 housing insert

45 flange

46 counter fastening element

48 protrusion

49 recess

50 counter coding

51 counter coding feature

52 counter coding protrusion

53 counter coding recess

55 electric counter coding feature

56 conducting path

57 conducting path

60 housing component

61 sidewall

62 distal end

64 housing receptacle

65 end face

66 counter fastening element

68 recess

69 protrusion

70 counter coding

71 counter coding feature

72 counter coding recess

73 counter coding protrusion

75 electric counter coding feature

76 electrical contact

77 electrical contact

78 resistor

79 neck portion

80 surface mounted resistor

100 add-on device

101 housing

102 fastener

110 adapter

112 adapter sleeve

114 extension

115 bridging portion

140 interface

141 sidewall

142 longitudinal end

144 interface receptacle

145 stepped portion

146 fastening element

147 snap element

148 recess

149 protrusion

150 coding

151 coding feature

152 coding protrusion

154 ring

155 electric coding

156 gear wheel

157 teeth

158 teeth

160 interface

161 sidewall

162 longitudinal end

164 interface insert

165 stepped portion

166 fastening element

167 snap element

168 protrusion

169 recess

169a groove portion

169b groove portion

170 coding

171 coding feature

173 coding protrusion

180 code recognition unit

181 electrical contact

182 electrical contact

183 electrical contact

184 electrical contact

190 coding configurator

191 actuator

192 latch

193 return spring

194 follower

195 follower sleeve

196 rotary encoding

197 stop

198 abutment

199 stop

200 logic circuit

201 printed circuit board

202 processor

204 switch

206 battery

208 sensor

210 communication interface

212 input

214 transceiver

216 speaker

218 display

220 memory

222 magnetic sensor

224 optical sensor

226 camera

228 rotary sensor

300 external electronic device

310 communication interface

316 speaker

318 display
1-16. (canceled)

17. An add-on device for attachment to an injection device, wherein the injection device comprises a device housing and a drive mechanism, wherein the device housing is configured to accommodate a medicament container filled with a medicament, wherein the drive mechanism is configured to operably engage with the medicament container to dispense a dose of the medicament, and wherein the injection device comprises at least one code, the add-on device comprising:
a fastener for attaching the add-on device to the device housing;
a code recognition unit operable to detect and/or to capture the at least one code of the injection device;
a logic circuit;
a processor; and
a communication interface operable to execute at least one electronically implemented function of the add-on device for assisting a user in operating the injection device and/or for monitoring operation of the injection device, wherein at least one of an execution and a calibration of the at least one electronically implemented function is modifiable by a signal obtainable from the code recognition unit in response to detecting and/or to capturing the at least one code of the injection device.

18. The add-on device of claim 17, wherein the at least one code is indicative of at least one of:
a type of the medicament,
a concentration of a pharmaceutically active substance of the medicament,
a size of the medicament container,
a type of the drive mechanism,
a manufacturing date of the medicament, or
an expiry date of the medicament.

19. The add-on device of claim 17, further comprising at least one sensor connected to the processor and operable to detect and/or to quantitatively measure at least one of setting of a dose or dispensing of a dose of the medicament by the injection device.

20. The add-on device of claim 19, wherein the processor is operable to calculate a size of a dose based on signals obtained from the sensor and based on the detected or captured code.

21. The add-on device of claim 17, wherein the processor is operable to generate at least one of an audible or visible output in response to detecting or to capturing the at least one code of the injection device.

22. The add-on device of claim 17, wherein the communication interface comprises a transceiver to communicate with an external electronic device.

23. The add-on device of claim 17, wherein the fastener comprises a mounting adapter, the mounting adapter comprising a first connecting interface and a second connecting interface, wherein the first connecting interface is connectable to a first housing component of the device housing and wherein the second connecting interface is connectable to a second housing component of the device housing.

24. The add-on device of claim 23, wherein at least one the first connecting interface and the second connecting interface comprises a coding configured to cooperate with a first mechanical counter coding of the first housing component or to cooperate with a second mechanical counter coding of the second housing component.

25. The add-on device of claim 24, wherein one of the first connecting interface and the second connecting interface comprises the mechanical coding, which is a reconfigurable mechanical coding, and wherein the other one of the first connecting interface and the second connecting interface comprises the code recognition unit.

26. The add-on device of claim 25, further comprising a coding configurator operably connected to the code recognition unit and operable to configure or to adapt the reconfigurable mechanical coding based on the code detected and/or captured by the code recognition unit.

27. The add-on device of claim 26, wherein the coding configurator comprises an electromechanical actuator mechanically engaged with a mechanical coding feature of the reconfigurable mechanical coding and operable to move the mechanical coding feature between at least a first coding position and a second coding position.

28. The add-on device of claim 26, wherein at least one of the code recognition unit and the coding configurator comprises a follower movably arranged in or on a housing of the add-on device, wherein the follower is operably engageable with one of the first mechanical counter coding provided on the first housing component and the second mechanical counter coding provided on the second housing component and wherein the follower is operably engaged with the configurable mechanical coding.

29. The add-on device of claim 28, wherein the configurable mechanical coding is connected to the follower, which is movable between at least a first follower position and a second follower position through a mechanical interaction with one of the first mechanical counter coding and the second mechanical counter coding.

30. An injection device for injecting a dose of a medicament, the injection device comprising:
a device housing configured to accommodate a medicament container filled with a medicament;
a drive mechanism configured to operably engage with the medicament container to dispense a dose of the medicament; and
at least one code on or in the device housing detectable and/or capturable by a code recognition unit of an add-on device comprising:
a fastener for attaching the add-on device to the device housing,
a code recognition unit operable to detect and/or to capture the at least one code of the injection device,
a logic circuit; a processor, and
a communication interface operable to execute at least one electronically implemented function of the add-on device for assisting a user in operating the injection device and/or for monitoring operation of the injection device, wherein at least one of an execution and a calibration of the at least one electronically implemented function is modifiable by a signal obtainable from the code recognition unit in response to detecting and/or to capturing the at least one code of the injection device.

31. The injection device of claim 30, wherein the at least one code is indicative of at least one of:
a type of the medicament,
a concentration of a pharmaceutically active substance of the medicament,
a size of the medicament container,
a type of the drive mechanism,
a manufacturing date of the medicament, or
an expiry date of the medicament.

32. The injection device of claim 30, wherein the drive mechanism comprises a piston rod comprising a pressure piece at a distal end.

33. The injection device of claim 30, wherein the fastener comprises a mounting adapter, the mounting adapter comprising a first connecting interface and a second connecting interface, wherein the first connecting interface is connectable to a first housing component of the device housing and wherein the second connecting interface is connectable to a second housing component of the device housing.

34. A drug delivery system comprising:
an injection device for injecting a dose of a medicament, the injection device comprising:
a device housing configured to accommodate a medicament container filled with a medicament;
a drive mechanism configured to operably engage with the medicament container to dispense a dose of the medicament;
at least one code on or in the device housing detectable and/or capturable by a code recognition unit of an add-on device; and
the add-on device, the add-on device comprising:
a fastener for attaching the add-on device to the device housing,
a code recognition unit operable to detect and/or to capture the at least one code of the injection device,
a logic circuit; a processor, and
a communication interface operable to execute at least one electronically implemented function of the add-on device for assisting a user in operating the injection device and/or for monitoring operation of the injection device, wherein at least one of an execution and a calibration of the at least one electronically implemented function is modifiable by a signal obtainable from the code recognition unit in response to detecting and/or to capturing the at least one code of the injection device.

35. A method of configuring an add-on device for attachment to an injection device, wherein the injection device comprises a device housing and a drive mechanism, wherein the device housing is configured to accommodate a medicament container filled with a medicament, wherein the drive mechanism is configured to operably engage with the medicament container to dispense a dose of the medicament, and wherein the injection device comprises at least one code, the method of configuring the add-on device comprising:
fastening and/or attaching the add-on device to the device housing,
detecting and/or capturing the at least one code of the injection device,
modifying at least one of an execution and a calibration of at least one electronically implemented function of the add-on device based on the detected or captured code of the injection device.

36. The method of claim 35, further comprising:
quantitatively measuring at least one of setting of a dose and dispensing of a dose of the medicament by the injection device by calculating a size of a dose based on signals obtained from a sensor and based on detecting and/or capturing the at least one code.
